(19) United States
(12) Patent Application Publication
(10) Pub. No.: US 2005/0244976 A1
(43) Pub. Date: Nov. 3, 2005

(54) Methods for detecting anionic and non-anionic compositions using carbocyanine dyes

(76) Inventors: Kyle Richard Gee, Springfield, OR (US); Wayne Forrest Patton, Newton, MA (US)

Correspondence Address: KOREN ANDERSON;MOLECULAR PROBES, INC., 29851 WILLOW CREEK ROAD, EUGENE, OR, 97402-9132, US

(21) Appl. No.: 11/063,707

(22) Filed: Feb. 22, 2005

Related U.S. Application Data
(60) Provisional application No. 60/546,663, filed on Feb. 20, 2004.

(51) Int. Cl. G01N 33/00; C07D 403/06

(52) U.S. Cl. 436/86; 548/305.7

(57) ABSTRACT

The present invention relates to methods of detecting anionic proteins in a sample with fluorescent carbocyanine dye compounds. The invention also describes methods of simultaneously detecting anionic and non-anionic proteins in a sample with discrete fluorescent signals produced by carbocyanine dye compounds. The invention is of use in a variety of fields including immunology, diagnostics, molecular biology and fluorescence based assays.

### CROSS-REFERENCE TO RELATED APPLICATIONS

This application claims priority of U.S. Ser. No. 60/546,663, filed Feb. 20, 2004, which disclosure is herein incorporated by reference.

### FIELD OF THE INVENTION

The present invention relates to methods of detecting anionic proteins in a sample with fluorescent carbocyanine dye compounds. The invention is of use in a variety of fields including immunology, diagnostics, proteomics, molecular biology and fluorescence based assays.

### BACKGROUND OF THE INVENTION

For decades, polyacrylamide gel electrophoresis and related blotting techniques have formed the core technologies for protein analysis. Traditionally, these technologies have been paired with chromogenic dye-based protein detection techniques, such as silver or Coomassie brilliant blue staining. Over the years, more specialized protein detecting stains were developed which could distinguish between subclasses of proteins or subproteomes. One of these compounds is (1-ethyl-2-[3-(1-ethyl-naphthol[1,2-d]thiazolin-2-ylidene)-2-methylpropenyl]naphthol[1,2-d]thiazolium bromide), alternatively known as STAINS-ALL. This compound is capable of preferentially staining anionic proteins blue. Examples of anionic proteins include phosphoproteins, sulfoproteins, calcium binding proteins, and sialoglycoproteins. In addition, STAINS-ALL is also capable of simultaneously staining the remaining non-anionic proteins red. This dual-staining capability, in theory, allowed researchers to gain more information from a single step, thus reducing labor-intensive multiple stain techniques.

In practice, however, STAINS-ALL has several inadequacies. First, it is less sensitive than Coomassie brilliant blue by an order of magnitude and also several orders of magnitude less sensitive than ³²P-autoradiography. This means that very large amounts of protein are required in order to obtain a detectable signal with the stain. Second, STAINS-ALL, as a colorimetric stain, is characterized by a very limited linear dynamic range, making protein quantitation more problematic. Third, STAINS-ALL is a light sensitive stain, and thus special precautions must be taken in order to ensure it does not degrade.

Fluorescence-based approaches to protein staining are a powerful alternative to colorimetric stains since the linear dynamic range of detection using fluorescent stains is usually superior to colorimetric stains. With the rapid growth of proteomics, new, highly quantitative protein staining techniques employing fluorescent molecules in electrophoresis gels are highly desired and increasingly gaining popularity. Among the desired protein staining techniques are those that preferentially stain anionic proteins, as well as those that possess dual-staining ability. The present invention addresses these and other problems.

### BRIEF SUMMARY OF THE INVENTION

In one embodiment is provided methods for detecting anionic proteins in a sample with carbocyanine dye compounds. The invention also describes methods of simultaneously detecting anionic and non-anionic proteins in a sample with discrete fluorescent signals produced by carbocyanine dye compounds. The invention is of use in a variety of fields including immunology, diagnostics, molecular biology and fluorescence based assays.

Thus, in a first aspect, the present invention provides a method for detecting the presence of an anionic protein and the presence of a non-anionic protein in a sample. The method includes contacting the sample with a compound having the following formula: 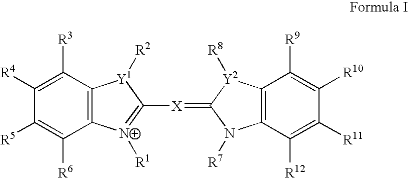
in which Y¹ and Y² are independently selected from S, O, N, and CR¹⁹. X is a member selected from 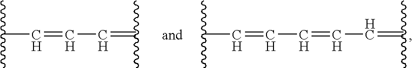
wherein at most one of said H is replaced with 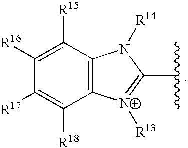
R¹, R², R⁷, R⁸, R¹³, and R¹⁴ are members independently selected from H and substituted or unsubstituted alkyl. R³, R⁴, R⁵, R⁶, R⁹, R¹⁰, R¹¹, R¹², R¹⁵, R¹⁶, R¹⁷, R¹⁸, and R¹⁹ are members independently selected from H, OH, NH₂, NO₂, —SO₂NH₂, nitro, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. R³ and R⁴, or R⁴ and R⁵, or R⁵ and R⁶, or R⁹ and R¹⁰, or R¹⁰ and R¹¹, or R¹¹ and R¹², or R¹⁵ and R¹⁶, or R¹⁶ and R¹⁷, or R¹⁷ and R¹⁸ together with the atoms to which they are joined, form a ring which is a 5-, 6- or 7-membered cycloalkyl, a substituted 5-, 6- or 7-membered cycloalkyl, a 5-, 6- or 7-membered heterocycloalkyl, a substituted 5-, 6- or 7-membered heterocycloalkyl, a 5-, 6- or 7-membered aryl, a substituted 5-, 6- or 7-membered aryl, a 5-, 6- or 7-membered heteroaryl, or a substituted 5-, 6- or 7-membered heteroaryl. The product of this contacting is then incubated for a sufficient amount of time to allow the compound to associate with a protein selected from the anionic protein and the non-anionic protein. Then, the product of this step is illuminated with a first appropriate wavelength whereby the presence of said anionic protein in said sample is determined. Next, the product of this step is illuminated with a second appropriate wavelength whereby the presence of said non-anionic protein in said sample is determined.

In a second aspect, the present invention provides a method for detecting an anionic protein in a sample. This method comprises contacting said sample with a compound which has a formula selected from: 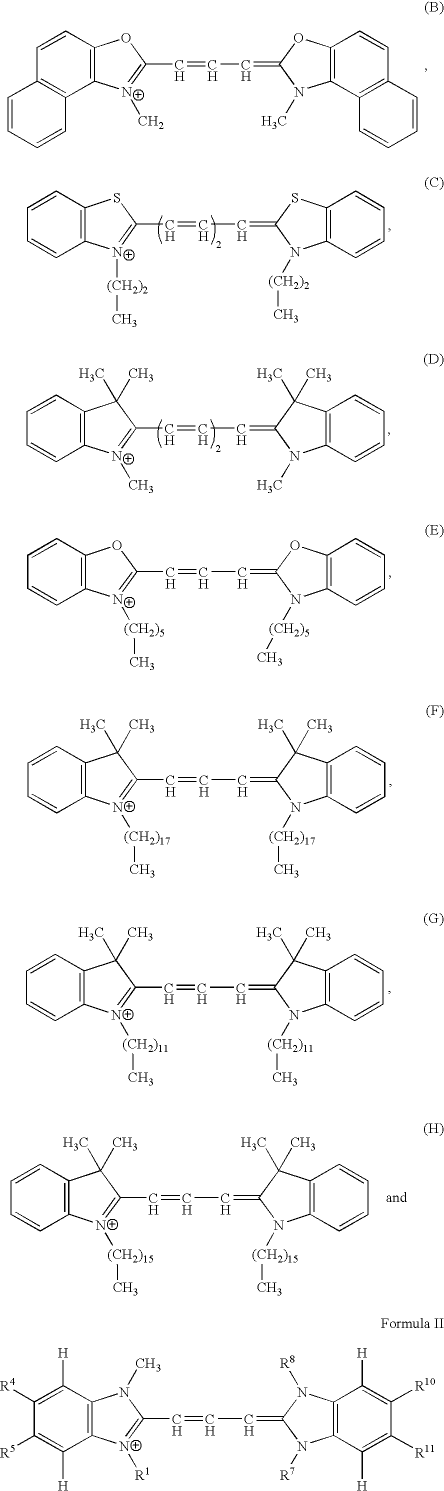
in which R¹, R², R⁷, and R⁸ are substituted or unsubstituted alkyl. R⁴, R⁵, R¹⁰, and R¹¹ are halogen. The product of step a) is incubated for sufficient time to allow said compound to associate with said anionic protein. The sample is then illuminated with a first appropriate wavelength whereby the presence of said anionic protein in said sample is determined.

In a third aspect, the invention provides a kit which comprises a compound that has a formula selected from: 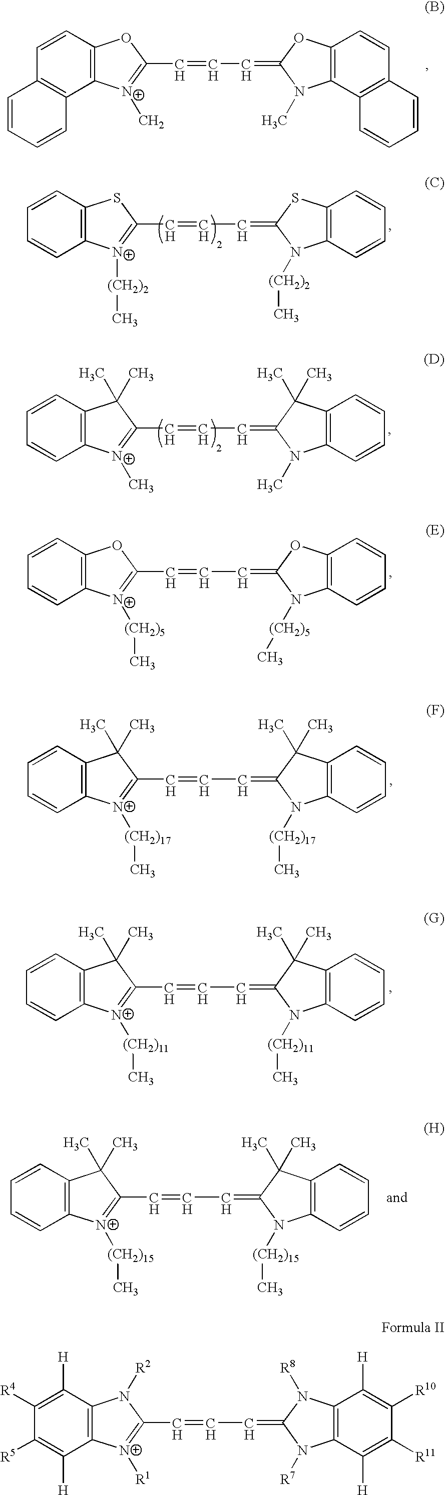
in which R¹, R², R⁷, and R⁸ are substituted or unsubstituted alkyl; and R⁴, R⁵, R¹⁰, and R¹¹ are halogen. The kit also provides instructions on the use of the compound.

### BRIEF DESCRIPTION OF THE DRAWINGS

FIG. 1 is a representation of the fluorescent intensity of bovine serum albumin and chicken ovalbumin as a function of distance for an electrophoretic separation. The separation was stained with compound (A) and SYPRO Ruby dye. See Example 2

FIG. 2 is a representation of the fluorescent intensity of pepsin and α-casein as a function of distance for an electrophoretic separation. The separation was stained with compound (A) and Stains All. See, Example 7

FIG. 3 is a fluorescence intensity of (A) α-casein, (B) β-casein, (c) Ovalbumin, (D) Pepsin, (E) Soybean trypsin inhibitor, (F) α1 acid glycoprotein and (G) BSA in solution with Compound A. Increasing concentrations of Compound A were added to individual cuvettes demonstrating an increase in fluorescent intensity with increasing concentrations of anionic proteins and no increase with non-anionic proteins. See, Example 6.

### DETAILED DESCRIPTION OF THE INVENTION

Introduction

There is a continuous and expanding need for rapid, highly specific methods of detecting and quantifying chemical, biochemical and biological analytes in research and diagnostic mixtures. Of particular value are methods for measuring small quantities of nucleic acids, peptides (e.g., enzymes), pharmaceuticals, metabolites, microorganisms and other materials of diagnostic value. Examples of such materials include narcotics and poisons, drugs administered for therapeutic purposes, hormones, pathogenic microorganisms and viruses, antibodies, and enzymes and nucleic acids, particularly those implicated in disease states.

One method of detecting an analyte relies on directly or indirectly labeling the analyte or other component of the analysis mixture with a fluorescent species. Fluorescent labels have the advantage of requiring few precautions in handling, and being amenable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling.

As discussed herein, the present invention provides methods of using carbocyanine dyes in order to detecting the presence of anionic proteins in a sample. This technology finds use in a variety of analytical and diagnostic techniques.

Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a carbocyanine compound” includes a plurality of proteins and reference to “a protein” includes a plurality of proteins and the like.

Unless defined otherwise, all technical and scientific terms used herein have the same meaning as commonly understood by one of ordinary skill in the art to which this invention is related. The following terms are defined for purposes of the invention as described herein.

The symbol [symbol] , whether utilized as a bond or displayed perpendicular to a bond indicates the point at which the displayed moiety is attached to the remainder of the molecule, solid support, etc.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose an appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom which is a member selected from the group consisting of O, N, Si, P and S, and wherein the nitrogen, phosphorous and sulfur atoms are optionally oxidized, and the nitrogen heteroatom is optionally be quaternized. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 3 rings), which are fused together or linked covalently. The term “heteroaryl” refers to aryl groups (or rings) that contain from one to four heteroatoms which are a member selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″ R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or N+RR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —N+RR′R″ and its biologically compatible anionic counterions.

The term “anionic protein”, as used herein, refers to a protein that possesses a net negative charge overall or in select regions along the polypeptide backbone in the environment in which it is located. Thus, a protein with overall isoelectric point of 1-6 would qualify, as well as a protein with a calcium-binding pocket containing numerous aspartic acid residues, despite the overall isoelectric point of the protein.

The term “aqueous solution” as used herein refers to a solution that is predominantly water and retains the solution characteristics of water. Where the aqueous solution contains solvents in addition to water, water is typically the predominant solvent.

The term “calcium-binding protein”, as used herein, refers to a protein that comprises a site in which an interaction with a calcium atom can occur. There are different classes of calcium-binding proteins and many of the more common ones are described in: Guidebook to Calcium-Binding Proteins, ed. by Marco R. Celio, co-edited by Thomas Pauls and Beat Schwaller, Oxford University Press, 1996. Examples of calcium-binding proteins include troponin C, alpha-actinin, calcineurin, calpains, SPARC and calmodulin.

The term “detectable response” as used herein refers to an occurrence of or a change in, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters.

The term “dye” as used herein refers to a carbocyanine compound that emits light to produce an observable detectable signal.

The term “fluorophore” or “fluorogenic” as used herein refers to a carbocyanine composition that is inherently fluorescent or demonstrates a change in fluorescence upon binding to a biological compound or metal ion, or metabolism by an enzyme. Fluorophores may be substituted to alter the solubility, spectral properties or physical properties of the fluorophore.

The term “carrier molecule” as used herein refers to a compound of the present invention that is covalently bonded to a biological or a non-biological component. Such components include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof.

The term “Linker” or “L”, as used herein, refers to a single covalent bond or a series of stable covalent bonds incorporating 1-20 nonhydrogen atoms selected from the group consisting of C, N, O, S and P that covalently attach the fluorogenic or fluorescent compounds to another moiety such as a chemically reactive group or a biological and non-biological component. Exemplary linking members include a moiety that includes —C(O)NH—, —C(O)O—, —NH—, —S—, —O—, and the like. A “cleavable linker” is a linker that has one or more cleavable groups that may be broken by the result of a reaction or condition. The term “cleavable group” refers to a moiety that allows for release of a portion, e.g., a fluorogenic or fluorescent moiety, of a conjugate from the remainder of the conjugate by cleaving a bond linking the released moiety to the remainder of the conjugate. Such cleavage is either chemical in nature, or enzymatically mediated. Exemplary enzymatically cleavable groups include natural amino acids or peptide sequences that end with a natural amino acid.

In addition to enzymatically cleavable groups, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available.

An exemplary cleavable group, an ester, is cleavable group that may be cleaved by a reagent, e.g. sodium hydroxide, resulting in a carboxylate-containing fragment and a hydroxyl-containing product.

The linker can be used to attach the compound to another component of a conjugate, such as a targeting moiety (e.g., antibody, ligand, non-covalent protein-binding group, etc.), an analyte, a biomolecule, a drug and the like.

The term “non-anionic protein”, as used herein, refers to a protein that possess either no charge or a net positive charge in the environment in which it is located. Typically, a protein with isoelectric point higher than 6 and without clusters of anionic amino acids in its linear sequence would qualify as a non-anionic protein.

The term “phosphoprotein”, as used herein, refers to a polypeptide possessing one or more phosphate or phosphate analog moieties each attached to such polypeptide by a single ester bond or inorganic phosphate. Phosphate analogs include, without limitation, thiophosphate, boronophosphate, phosphoramide, H-phosphonate, alkylphosphonate, phosphorothioate, phosphorodithioate and phosphorofluoridate. Most known phosphate compounds, and subsequently the phosphoproteins, can be categorized into one of three groups; 1) individual phosphate groups (e.g., inorganic phosphate or a phosphate group (PO₃) on a protein or peptide); 2) multiple-linked phosphate group (e.g., pyrophosphate or a nucleotide such as ATP); or 3) bridging phosphate group (i.e., nucleic acids). For the purposes of the present invention, phosphoproteins do not include molecules in the third group, e.g., DNA or RNA. Typically, phosphoproteins and phosphopeptides are phosphorylated post-translationally on the tyrosine, serine or threonine amino acid residues. Other phosphorylated amino acid residues in peptides and proteins include 1-phospho-histidine, 3-phospho-histidine, phospho-aspartic acid, phospho-glutamic acid and less commonly N^{ε}-phospho-lysine, N^{ω}-phospho-arginine and phospho-cysteine (Kaufmann, et al (2001) Proteomics 1: 194-199; Yan, J., Packer, N., Gooley, A. and Williams, K. (1998) J. Chromatograph. A 808: 23-41). Thus, a phosphorylated protein or peptide typically comprises at least one of these amino acid residues. Phosphoproteins also include phosphorylated proteins that incorporate other non-peptide regions such as lipids or carbohydrates, e.g., lipoproteins and lipopolysaccharides. In addition, the lipid or carbohydrate residues of the proteins can be phosphorylated instead or in combination with the tyrosine, serine or threonine amino acid residues of the proteins and peptides such as a phosphomannose-modified or N-acetylglucosamine-1-phosphate modified protein. Other modifications include a pyridoxal phosphate Schiff base to the epsilon-amino group of lysine, and an O-pantetheine phosphorylation of serine residue. The gamma phosphate of nucleotide triphosphates is also detectable using the methods of this invention, making photolabeled proteins and peptides detectable by this procedure.

The terms “protein” and “polypeptide” are used herein in a generic sense to include polymers of amino acid residues of any length. The term “peptide” is used herein to refer to polypeptides having less than 250 amino acid residues, typically less than 100 amino acid residues. The terms apply to amino acid polymers in which one or more amino acid residues are an artificial chemical analogue of a corresponding naturally occurring amino acid, as well as to naturally occurring amino acid polymers.

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as carboxylic acid or succinimidyl ester, on the compounds of the present invention that is capable of chemically reacting with a functional group on a different compound to form a covalent linkage. Reactive groups generally include nucleophiles, electrophiles and photoactivatable groups.

Exemplary reactive groups include, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds., Organic Functional Group Preparations, Academic Press, San Diego, 1989).

The term “photoactivatable reactive group” as used herein refers to a chemical moiety that becomes chemically active by exposure to an appropriate wavelength, typically a UV wavelength. Once activated the reactive group is capable of forming a covalent bond with a proximal moiety on a biological or non-biological component. In the instant case, the carbocyanine dyes may contain a photoactivatable group that can form a covalent bond with an anionic protein when brought within proximity by the formation of the ternary complex and activated by an appropriate wavelength. Photoactivatable groups include, but are not limited to, benzbphenones, aryl azides and diazirines.

“rt”, as used herein, refers to room temperature.

The term “sialoglycoproteins”, as used herein, refers to a glycoprotein modified on the glycan moiety with one or more sialic acid residues.

The term “sulfoprotein” as used herein, refers to a protein modified at tyrosine residues with a sulfate group or alternatively a glycoprotein modified on the glycan moiety with sulfate residues.

The term “sample” as used herein refers to any material that may contain an anionic or non-anionic protein. Typically, the sample is a live cell, a biological fluid that comprises endogenous host cell proteins, nucleic acid polymers, nucleotides, oligonucleotides, peptides and buffer solutions. The sample may be in an aqueous solution, a viable cell culture or immobilized on a solid or semi solid surface such as a polyacrylamide gel, membrane blot or on a microarray.

The Compounds

The methods of the present invention involve compounds that are useful for the detection of anionic proteins in a sample. In an exemplary embodiment, these compounds can simultaneously detect the presence of non-anionic proteins in a sample. The components of the compounds used in the methods of the invention are described in greater detail below.

a) Carbocyanine Dyes

a) i) Carbocyanine Dyes for Detecting Anionic Proteins

The carbocyanine dyes that are useful for the detection of anionic proteins are: 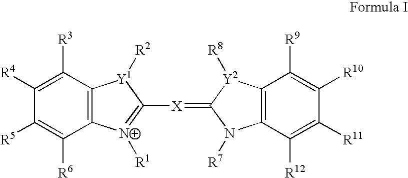
in which Y¹ and Y² are independently selected from S, O, N, and CR¹⁹. X is a member selected from 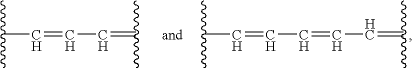
wherein at most one of said H is replaced with 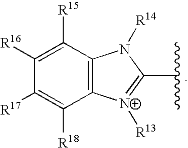
R¹, R², R⁷, R⁸, R¹³, and R¹⁴ are members independently selected from H and substituted or unsubstituted alkyl. R³, R⁴, R⁵, R⁶, R⁹, R¹⁰, R¹¹, R¹², R¹⁵, R¹⁶, R¹⁷, R¹⁸, and R¹⁹ are members independently selected from H (hydrogen), OH, NH₂, NO₂, —SO₂NH₂, nitro, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. R³ and R⁴, or R⁴ and R⁵, or R⁵ and R⁶, or R⁹ and R¹⁰, or R¹⁰ and R¹¹, or R¹¹ and R¹², or R¹⁵ and R¹⁶, or R¹⁶ and R¹⁷, or R¹⁷ and R¹⁸ together with the atoms to which they are joined, form a ring which is a 5-, 6- or 7-membered cycloalkyl, a substituted 5-, 6- or 7-membered cycloalkyl, a 5-, 6- or 7-membered heterocycloalkyl, a substituted 5-, 6- or 7-membered heterocycloalkyl, a 5-, 6- or 7-membered aryl, a substituted 5-, 6- or 7-membered aryl, a 5-, 6- or 7-membered heteroaryl, or a substituted 5-, 6- or 7-membered heteroaryl.

In an exemplary embodiment, the carbocyanine dyes are members selected from: 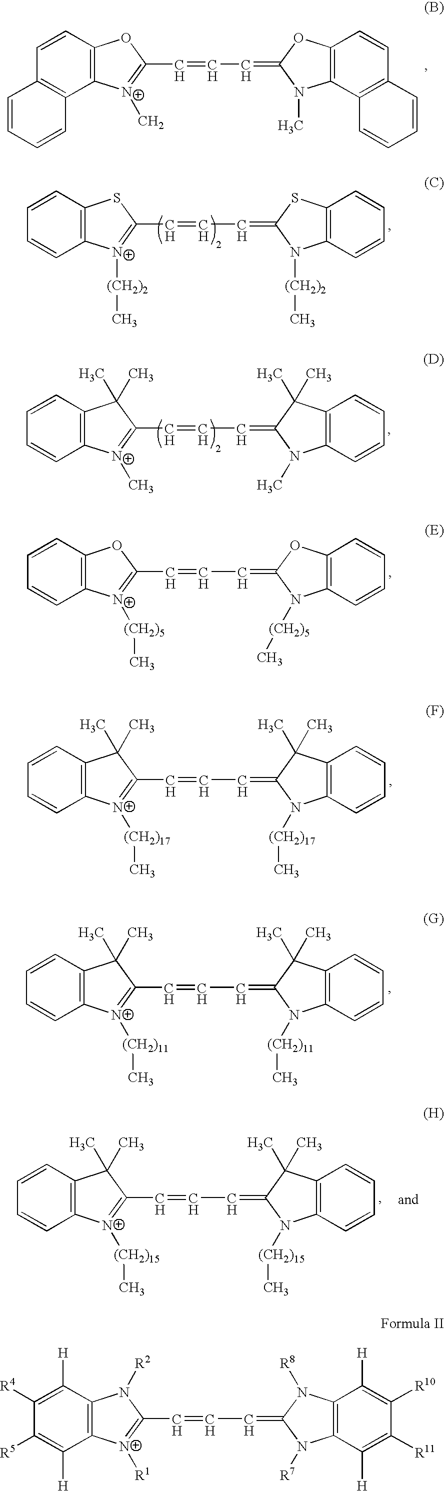
in which R¹, R², R⁷, and R⁸ are substituted or unsubstituted alkyl; and R⁴, R⁵, R¹⁰, and R¹¹ are halogen.

In an exemplary embodiment, the compound is 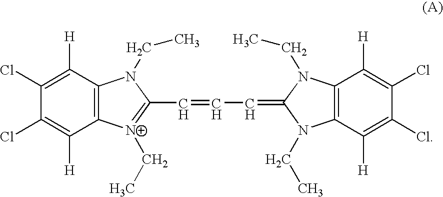
a) ii) Carbocyanine Dyes for Simultaneously Detecting Anionic and Non-Anionic Proteins

Depending on their concentration in a certain area, carbocyanine dyes can exist in two different states. At low concentration, non-covalent interactions between carbocyanine dyes in a material are minimized, enabling the carbocyanine dyes to exist in a “monomer” state. At higher concentrations, however, non-covalent interactions between carbocyanine dyes increase, causing a portion of the carbocyanine dye molecules to exist in an “aggregate” state. One of the changes to be expected from a “monomer” state to an “aggregate” state is an alteration of the optical properties of the two materials. However, for most carbocyanine dyes, the optical changes between a “monomer” and “aggregate” format are difficult to perceive. Quite surprisingly, it has been discovered that the “monomer” and “aggregate” states in certain carbocyanine dyes are both pronounced in the fluorescence detection regime, and discrete from one another in order to be separately detectable. Even more surprising is the finding that certain of these carbocyanine dyes interact in a “monomer” state with non-anionic proteins, while interacting in an “aggregate” state with anionic proteins. For example, as shown in Example 1, compound (A) fluoresces red when staining anionic proteins, and green when staining non-anionic proteins.

The carbocyanine dyes that are useful for simultaneously detecting anionic and non-anionic proteins are: 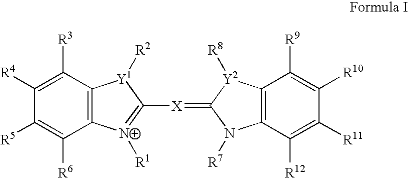
in which Y¹ and Y² are independently selected from S, O, N, and CR¹⁹. X is a member selected from 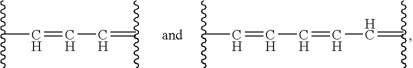
wherein at most one of said H is replaced with 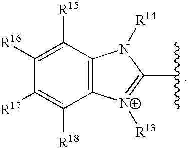
R¹, R², R⁷, R⁸, R¹³ and R¹⁴ are members independently selected from H and substituted or unsubstituted alkyl. R³, R⁴, R⁵, R⁶, R⁹, R¹⁰, R¹¹, R¹², R¹⁵, R¹⁶, R¹⁷, R¹⁸, and R¹⁹ are members independently selected from H, OH, NH₂, NO₂, —SO₂NH₂, nitro, cyano, halogen, substituted or unsubstituted alkyl, substituted or unsubstituted heteroalkyl, substituted or unsubstituted 3- to 7-membered cycloalkyl, substituted or unsubstituted 5- to 7-membered heterocycloalkyl, substituted or unsubstituted aryl, and substituted or unsubstituted heteroaryl. R³ and R⁴, or R⁴ and R⁵, or R⁵ and R⁶, or R⁹ and R¹⁰, or R¹⁰ and R¹¹, or R¹¹ and R¹², or R¹⁵ and R¹⁶, or R¹⁶ and R¹⁷, or R¹⁷ and R¹⁸ together with the atoms to which they are joined, form a ring which is a 5-, 6- or 7-membered cycloalkyl, a substituted 5-, 6- or 7-membered cycloalkyl, a 5-, 6- or 7-membered heterocycloalkyl, a substituted 5-, 6- or 7-membered heterocycloalkyl, a 5-, 6- or 7membered aryl, a substituted 5-, 6- or 7-membered aryl, a 5-, 6- or 7-membered heteroaryl, or a substituted 5-, 6- or 7-membered heteroaryl.

In an exemplary embodiment, Y¹ and Y² are N.

In an exemplary embodiment, the compound is: 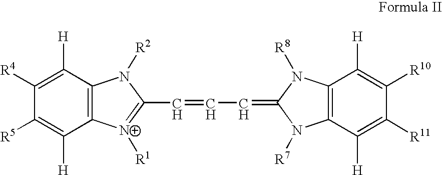
in which R¹, R², R⁷, and R⁸ are substituted or unsubstituted alkyl; and R⁴, R⁵, R¹⁰, and R¹¹ are halogen.

In another exemplary embodiment, the compound is: 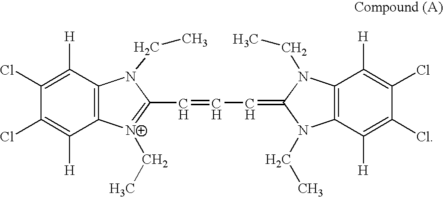
b) i) Reactive Groups, Carrier Molecules and Solid Supports

The present compounds, in certain embodiments, are chemically reactive wherein the compounds comprise a reactive group. In a further embodiment, the compounds comprise a carrier molecule or solid support. These substituents, reactive groups, carrier molecules, and solid supports, comprise a linker that is used to covalently attach the substituents to any of the moieties of the present compounds. The solid support, carrier molecule or reactive group may be directly attached (where linker is a single bond) to the moieties or attached through a series of stable bonds, as disclosed above.

Any combination of linkers may be used to attach the carrier molecule, solid support or reactive group and the present compounds together. The linker may also be substituted to alter the physical properties of the reporter moiety or chelating moiety, such as spectral properties of the dye. Examples of L include substituted or unsubstituted polyalkylene, arylene, alkylarylene, arylenealkyl, or arylthio moieties.

The linker typically incorporates 1-30 nonhydrogen atoms selected from the group consisting of C, N, O, S and P. The linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. Typically the linker incorporates less than 15 nonhydrogen atoms and are composed of any combination of ether, thioether, thiourea, amine, ester, carboxamide, sulfonamide, hydrazide bonds and aromatic or heteroaromatic bonds. Typically the linker is a combination of single carbon-carbon bonds and carboxamide, sulfonamide or thioether bonds. The bonds of the linker typically result in the following moieties that can be found in the linker: ether, thioether, carboxamide, thiourea, sulfonamide, urea, urethane, hydrazine, alkyl, aryl, heteroaryl, alkoxy, cycloalkyl and amine moieties. Examples of a linker include substituted or unsubstituted polymethylene, arylene, alkylarylene, arylenealkyl, and arylthio.

In one embodiment, the linker contains 1-6 carbon atoms; in another, the linker comprises a thioether linkage. Exemplary linking members include a moiety that includes —C(O)NH—, —C(O)O—, —NH—, —S—, —O—, and the like. In another embodiment, the linker is or incorporates the formula —(CH₂)_{d}(CONH(CH₂)ₑ)_{z}— or where d is an integer from 0-5, e is an integer from 1-5 and z is 0 or 1. In a further embodiment, the linker is or incorporates the formula —O—(CH₂)—. In yet another embodiment, the linker is or incorporates a phenylene or a 2-carboxy-substituted phenylene.

Any combination of linkers may be used to attach the reactive groups and the present compounds together, typically a compound of the present invention when attached to more than one reactive group will have one or two linkers attached that may be the same or different. The linker may also be substituted to alter the physical properties of the present compounds, such as solubility and spectral properties of the compound.

The reactive group can be bound to the carbocyanine dye at R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, or R¹⁹. In another exemplary embodiment, the reactive group can be bound to the carbocyanine dye at R¹, R², R⁷, R⁸, R¹³, or R¹⁴. In another exemplary embodiment, the reactive group can be bound to the carbocyanine dye at R³, R⁴, R⁵, R⁶, R⁹, R¹⁰, R¹¹, R¹², R¹⁵, R¹⁶, R¹⁷, R¹⁸, or R¹⁹.

In an exemplary embodiment, the compounds of the invention further comprise a reactive group which is a member selected from an acrylamide, an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine, a hydrazide, an imido ester, an isocyanate, an isothiocyanate, a maleimide, a phosphoramidite, a reactive platinum complex, a sulfonyl halide, a thiol group, and a photoactivatable group.

These reactive groups can be covalently attached either during or after the synthesis of the carbocyanine dyes in order to provide reactive group-containing-carbocyanine dyes. In this way, reactive group-containing-carbocyanine dyes can be covalently attached to a wide variety of carrier molecules or solid supports that contain or are modified to contain functional groups with suitable reactivity, resulting in chemical attachment of the components. In an exemplary embodiment, the reactive group of a compound of the invention and the functional group of the carrier molecule of solid support comprise electrophiles and nucleophiles that can generate a covalent linkage between them. Alternatively, the reactive group comprises a photoactivatable group, which becomes chemically reactive only after illumination with light of an appropriate wavelength. Typically, the conjugation reaction between the reactive group and the carrier molecule/solid support results in one or more atoms of the reactive group being incorporated into a new linkage attaching the carbocyanine dye to the carrier molecule/solid support. Selected examples of functional groups and linkages are shown in Table 1, where the reaction of an electrophilic group and a nucleophilic group yields a covalent linkage.

TABLE 1
Examples of some routes to useful covalent linkages with electrophile
and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| *Activated esters, as understood in the art, generally have the formula —COΩ, where Ω is good leaving group (e.g. oxysuccinimidyl (—OC₄H₄O₂) oxysulfosuccinimidyl (—OC₄H₃O₂— SO₃H), -1-oxybenzotriazolyl (—OC₆H₄N₃); | | |
| # or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, | | |
| # where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl). | | |

**Acyl azides can also rearrange to isocyanates

In one aspect, the compound comprises at least one reactive group that selectively reacts with an amine group. This amine-reactive group is selected from the group consisting of succinimidyl ester, sulfonyl halide, tetrafluorophenyl ester and iosothiocyanates. Thus, in one aspect, the present compounds form a covalent bond with an amine-containing molecule in a sample. In another aspect, the compound comprises at least one reactive group that selectively reacts with a thiol group. This thiol-reactive group is selected from the group consisting of maleimide, haloalkyl and haloacetamide (including any reactive groups disclosed in U.S. Pat. Nos. 5,362,628; 5,352,803 and 5,573,904).

Choice of the reactive group used to attach the compound of the invention to the substance to be conjugated typically depends on the reactive or functional group on the substance to be conjugated and the type or length of covalent linkage desired. The types of functional groups typically present on the organic or inorganic substances (biomolecule or non-biomolecule) include, but are not limited to, amines, amides, thiols, alcohols, phenols, aldehydes, ketones, phosphates, imidazoles, hydrazines, hydroxylamines, disubstituted amines, halides, epoxides, silyl halides, carboxylate esters, sulfonate esters, purines, pyrimidines, carboxylic acids, olefinic bonds, or a combination of these groups. A single type of reactive site may be available on the substance (typical for polysaccharides or silica), or a variety of sites may occur (e.g., amines, thiols, alcohols, phenols), as is typical for proteins.

Typically, the reactive group will react with an amine, a thiol, an alcohol, an aldehyde, a ketone, or with silica. Preferably, reactive groups react with an amine or a thiol functional group, or with silica. In one embodiment, the reactive group is an acrylamide, an activated ester of a carboxylic acid, an acyl azide, an acyl nitrile, an aldehyde, an alkyl halide, a silyl halide, an anhydride, an aniline, an aryl halide, an azide, an aziridine, a boronate, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as described in U.S. Pat. No. 5,714,327.

Where the reactive group is an activated ester of a carboxylic acid, such as a succinimidyl ester of a carboxylic acid, a sulfonyl halide, a tetrafluorophenyl ester or an isothiocyanates, the resulting compound is particularly useful for preparing conjugates of carrier molecules such as proteins, nucleotides, oligonucleotides, or haptens. Where the reactive group is a maleimide, haloalkyl or haloacetamide (including any reactive groups disclosed in U.S. Pat. Nos. 5,362,628; 5,352,803 and 5,573,904 (supra)) the resulting compound is particularly useful for conjugation to thiol-containing substances. Where the reactive group is a hydrazide, the resulting compound is particularly useful for conjugation to periodate-oxidized carbohydrates and glycoproteins, and in addition is an aldehyde-fixable polar tracer for cell microinjection. Where the reactive group is a silyl halide, the resulting compound is particularly useful for conjugation to silica surfaces, particularly where the silica surface is incorporated into a fiber optic probe subsequently used for remote ion detection or quantitation.

In a particular aspect, the reactive group is a photoactivatable group such that the group is only converted to a reactive species after illumination with an appropriate wavelength. An appropriate wavelength is generally a UV wavelength that is less than 400 nm. This method provides for specific attachment to only the target molecules, either in solution or immobilized on a solid or semi-solid matrix. In this way, present carbocyanine dye compounds that comprise a photoactivatable reactive group associate with anionic proteins and can be covalently conjugated to the proteins. Photoactivatable reactive groups include, without limitation, benzophenones, aryl azides and diazirines.

Preferably, the reactive group is a photoactivatable group, succinimidyl ester of a carboxylic acid, a haloacetamide, haloalkyl, a hydrazine, an isothiocyanate, a maleimide group, an aliphatic amine, a silyl halide, a cadaverine or a psoralen. More preferably, the reactive group is a succinimidyl ester of a carboxylic acid, a maleimide, an iodoacetamide, or a silyl halide. In a particular embodiment the reactive group is a succinimidyl ester of a carboxylic acid, a sulfonyl halide, a tetrafluorophenyl ester, an iosothiocyanates or a maleimide.

The selection of a covalent linkage to attach the reporter molecule to the carrier molecule or solid support typically depends on the chemically reactive group on the component to be conjugated. The discussion regarding reactive groups in the section immediately preceding is relevant here as well. Exemplary reactive groups typically present on the biological or non-biological components include, but are not limited to, amines, thiols, alcohols, phenols, aldehydes, ketones, phosphates, imidazoles, hydrazines, hydroxylamines, disubstituted amines, halides, epoxides, sulfonate esters, purines, pyrimidines, carboxylic acids, or a combination of these groups. A single type of reactive site may be available on the component (typical for polysaccharides), or a variety of sites may occur (e.g. amines, thiols, alcohols, phenols), as is typical for proteins. A carrier molecule or solid support may be conjugated to more than one reporter molecule, which may be the same or different, or to a substance that is additionally modified by a hapten. Although some selectivity can be obtained by careful control of the reaction conditions, selectivity of labeling is best obtained by selection of an appropriate reactive compound.

In another exemplary embodiment, the carbocyanine dye is covalently bound to a carrier molecule. If the compound has a reactive group, then the carrier molecule can alternatively be linked to the compound through the reactive group. The reactive group may contain both a reactive functional moiety and a linker, or only the reactive functional moiety.

A variety of carrier molecules are useful in the present invention. Exemplary carrier molecules include antigens, steroids, vitamins, drugs, haptens, metabolites, toxins, environmental pollutants, amino acids, peptides, proteins, nucleic acids, nucleic acid polymers, carbohydrates, lipids, and polymers.

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide. In another exemplary embodiment, at least one member selected from R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ comprise a carrier molecule. In another exemplary embodiment, at least one member selected from R¹, R², R⁷, R⁸, R¹³, and R¹⁴ comprise a carrier molecule. In another exemplary embodiment, at least one member selected from R³, R⁴, R⁵, R⁶, R⁹, R¹⁰, R¹¹, R¹², R¹⁵, R¹⁶, R¹⁷, R¹⁸, and R¹⁹ comprise a carrier molecule.

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide. In a preferred embodiment the carrier molecule is amino acid, a peptide, a protein, a polysaccharide, a nucleoside, a nucleotide, an oligonucleotide, a nucleic acid, a hapten, a psoralen, a drug, a hormone, a lipid, a lipid assembly, a tyramine, a synthetic polymer, a polymeric microparticle, a biological cell, cellular components, an ion chelating moiety, an enzymatic substrate or a virus. In another preferred embodiment, the carrier molecule is an antibody or fragment thereof, an antigen, an avidin or streptavidin, a biotin, a dextran, an antibody binding protein, a fluorescent protein, agarose, and a non-biological microparticle. Typically, the carrier molecule is an antibody, an antibody fragment, antibody-binding proteins, avidin, streptavidin, a toxin, a lectin, or a growth factor. Preferred haptens include biotin, digoxigenin and fluorophores.

Antibody binging proteins include, but are not limited to, protein A, protein G, soluble Fc receptor, protein L, lectins, anti-IgG, anti-IgA, anti-IgM, anti-IgD, anti-IgE or a fragment thereof.

In an exemplary embodiment, the enzymatic substrate is selected from an amino acid, peptide, sugar, alcohol, alkanoic acid, 4-guanidinobenzoic acid, nucleic acid, lipid, sulfate, phosphate, —CH₂OCOalkyl and combinations thereof. Thus, the enzyme substrates can be cleave by enzymes selected from the group consisting of peptidase, phosphatase, glycosidase, dealkylase, esterase, guanidinobenzotase, sulfatase, lipase, peroxidase, histone deacetylase, endoglycoceramidase, exonuclease, reductase and endonuclease.

In another exemplary embodiment, the carrier molecule is an amino acid (including those that are protected or are substituted by phosphates, carbohydrates, or C₁ to C₂₂ carboxylic acids), or a polymer of amino acids such as a, peptide or protein. In a related embodiment, the carrier molecule contains at least five amino acids, more preferably 5 to 36 amino acids. Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Preferred protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. Typically, the protein carrier molecule is an antibody, an antibody fragment, avidin, streptavidin, a toxin, a lectin, or a growth factor. Exemplary haptens include biotin, digoxigenin and fluorophores.

In another exemplary embodiment, the carrier molecule comprises a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955) or other linkage. In another exemplary embodiment, the nucleotide carrier molecule is a nucleoside or a deoxynucleoside or a dideoxynucleoside.

Exemplary nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides.

In another exemplary embodiment, the carrier molecule comprises a carbohydrate or polyol that is typically a polysaccharide, such as dextran, FICOLL, heparin, glycogen, amylopectin, mannan, inulin, starch, agarose and cellulose, or is a polymer such as a poly(ethylene glycol). In a related embodiment, the polysaccharide carrier molecule includes dextran, agarose or FICOLL.

In another exemplary embodiment, the carrier molecule comprises a lipid (typically having 6-25 carbons), including glycolipids, phospholipids, and sphingolipids. Alternatively, the carrier molecule comprises a lipid vesicle, such as a liposome, or is a lipoprotein (see below). Some lipophilic substituents are useful for facilitating transport of the conjugated dye into cells or cellular organelles.

Alternatively, the carrier molecule is cells, cellular systems, cellular fragments, or subcellular particles. Examples of this type of conjugated material include virus particles, bacterial particles, virus components, biological cells (such as animal cells, plant cells, bacteria, or yeast), or cellular components. Examples of cellular components that can be labeled, or whose constituent molecules can be labeled, include but are not limited to lysosomes, endosomes, cytoplasm, nuclei, histones, mitochondria, Golgi apparatus, endoplasmic reticulum and vacuoles.

In another embodiment the carrier molecule is a metal chelating moiety. While any chelator that binds a metal ion of interest and gives a change in its fluorescence properties is a suitable conjugate, preferred metal chelating moieties are crown ethers, including diaryidiaza crown ethers, as described in U.S. Pat. No. 5,405,975 to Kuhn et al. (1995); derivatives of 1,2-bis-(2-aminophenoxyethane)-N,N,N′,N′-tetraacetic acid (BAPTA), as described in U.S. Pat. No. 5,453,517 to Kuhn et al. (1995) (incorporated by reference) and U.S. Pat. No. 5,049,673 to Tsien et al. (1991); derivatives of 2-carboxymethoxy-aniline-N,N-diacetic acid (APTRA), as described by Ragu et al., Am. J. Physiol., 256: C540 (1989); and pyridyl-based and phenanthroline metal ion chelators, as described in U.S. Pat. No. 5,648,270 to Kuhn et al. (1997).

Fluorescent conjugates of metal chelating moieties possess utility as indicators for the presence of a desired metal ion. While fluorescent ion-indicators are known in the art, the incorporation of the fluorinated fluorogenic and fluorescent compounds of the present invention imparts the highly advantageous properties of the instant fluorophores onto the resulting ion indicator.

The ion-sensing conjugates of the invention are optionally prepared in chemically reactive forms and further conjugated to polymers such as dextrans to improve their utility as sensors as described in U.S. Pat. Nos. 5,405,975 and 5,453,517.

In another exemplary embodiment, the carrier molecule non-covalently associates with organic or inorganic materials. Exemplary embodiments of the carrier molecule that possess a lipophilic substituent can be used to target lipid assemblies such as biological membranes or liposomes by non-covalent incorporation of the dye compound within the membrane, e.g., for use as probes for membrane structure or for incorporation in liposomes, lipoproteins, films, plastics, lipophilic microspheres or similar materials.

In an exemplary embodiment, the carrier molecule comprises a specific binding pair member wherein the present compounds are conjugated to a specific binding pair member and are used to detect an analyte in a sample. Alternatively, the presence of the labeled specific binding pair member indicates the location of the complementary member of that specific binding pair; each specific binding pair member having an area on the surface or in a cavity which specifically binds to, and is complementary with, a particular spatial and polar organization of the other. Exemplary binding pairs are set forth in Table 2.

TABLE 2
Representative Specific Binding Pairs

| | |
|---|---|
| antigen | antibody |
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA)† |
| hormone | hormone receptor |
| ion | chelator |
| antibody | antibody-binding proteins |

*IgG is an immunoglobulin
†cDNA and cRNA are the complementary strands used for hybridization

b) iii) Solid Support

In an exemplary embodiment, the compounds of the invention are covalently bonded to a solid support. The solid support may be attached to the compound either through the carbocyanine dye, or through the reactive group, if present, or through a carrier molecule, if present. Even if a reactive group and/or a carrier molecule are present, the solid support may be attached through the carbocyanine dye. In another exemplary embodiment, at least one member selected from R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ is attached to a solid support. In another exemplary embodiment, at least one member selected from R¹, R², R⁷, R⁸, R¹³, and R¹⁴ is a solid support or is attached to a solid support. In another exemplary embodiment, at least one member selected from R³, R⁴, R⁵, R⁶, R⁹, R¹⁰, R¹¹, R¹², R¹⁵, R¹⁶, R¹⁷, R¹⁸, and R¹⁹ is a solid support or is attached to a solid support.

A solid support suitable for use in the present invention is typically substantially insoluble in liquid phases. Solid supports of the current invention are not limited to a specific type of support. Rather, a large number of supports are available and are known to one of ordinary skill in the art. Thus, useful solid supports include semi-solids, such as aerogels and hydrogels, resins, beads, biochips (including thin film coated biochips), multi-well plates (also referred to as microtitre plates), membranes, conducting and nonconducting metals and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the compounds of the invention. Useful reactive groups are disclosed above and are equally applicable to the solid support reactive functional groups herein.

A suitable solid phase support can be selected on the basis of desired end use and suitability for various synthetic protocols. For example, where amide bond formation is desirable to attach the compounds of the invention to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories).

Conjugates of components (carrier molecules or solid supports), e.g., drugs, peptides, toxins, nucleotides, phospholipids and other organic molecules are prepared by organic synthesis methods using the reactive dyes, are generally prepared by means well recognized in the art (Haugland, MOLECULAR PROBES HANDBOOK, supra, 2002). Conjugation to form a covalent bond may consist of simply mixing the reactive dyes of the present invention in a suitable solvent in which both the reactive compound and the substance to be conjugated are soluble. The reaction preferably proceeds spontaneously without added reagents at room temperature or below. For those reactive dyes that are photoactivated, conjugation is facilitated by illumination of the reaction mixture to activate the reactive dye. Chemical modification of water-insoluble substances, so that a desired dye-conjugate may be prepared, is preferably performed in an aprotic solvent such as dimethylformamide (DMF), dimethylsulfoxide (DMSO), acetone, ethyl acetate, toluene, or chloroform.

Preparation of peptide or protein conjugates typically comprises first dissolving the protein to be conjugated in aqueous buffer at about. 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive dye is then dissolved in a nonhydroxylic solvent (usually DMSO or DMF) in an amount sufficient to give a suitable degree of conjugation when added to a solution of the protein to be conjugated. The appropriate amount of compound for any protein or other component is conveniently predetermined by experimentation in which variable amounts of the dye are added to the protein, the conjugate is chromatographically purified to separate unconjugated compound and the compound-protein conjugate is tested in its desired application.

Following addition of the reactive compound to the component solution, the mixture may be incubated for a suitable period (typically about 1 hour at room temperature to several hours on ice), the excess unreacted compound is removed by gel filtration, dialysis, HPLC, adsorption on an ion exchange or hydrophobic polymer or other suitable means. The conjugate is used in solution or lyophilized. In this way, suitable conjugates can be prepared from antibodies, antibody fragments, avidins, lectins, enzymes, proteins A and G, cellular proteins, albumins, histones, growth factors, hormones, and other proteins. The approximate degree of substitution is determined from the long wavelength absorption of the compound-protein conjugate by using the extinction coefficient of the un-reacted compound at its long wavelength absorption peak, the unmodified protein's absorption peak in the ultraviolet and by correcting the UV absorption of the conjugate for absorption by the compound in the UV.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides or multiple types of reactive sites (e.g. amines, thiols, alcohols, phenols) may be available, as is typical for proteins. Selectivity of labeling is best obtained by selection of an appropriate reactive dye. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions.

When modifying polymers with the compounds, an excess of the compound is typically used, relative to the expected degree of dye substitution. Any residual, un-reacted compound or hydrolysis product is typically removed by dialysis, chromatography or precipitation. Presence of residual, unconjugated compound can be detected by thin layer chromatography using a solvent that elutes the compound away from its conjugate. In all cases it is usually preferred that the reagents be kept as concentrated as practical so as to obtain adequate rates of conjugation.

In an exemplary embodiment, the conjugate is associated with an additional substance that binds either to the compound or the labeled component through noncovalent interaction. In another exemplary embodiment, the additional substance is an antibody, an enzyme, a hapten, a lectin, a receptor, an oligonucleotide, a nucleic acid, a liposome, or a polymer. The additional substance is optionally used to probe for the location of the conjugate, for example, as a means of enhancing the signal of the conjugate.

c) Synthesis

c) i) Methods of Attaching Photoactivatable Groups to a Carbocyanine Dye

In Scheme 1, a general preparatory synthesis for a photoactivatable version of compound (A) is presented.

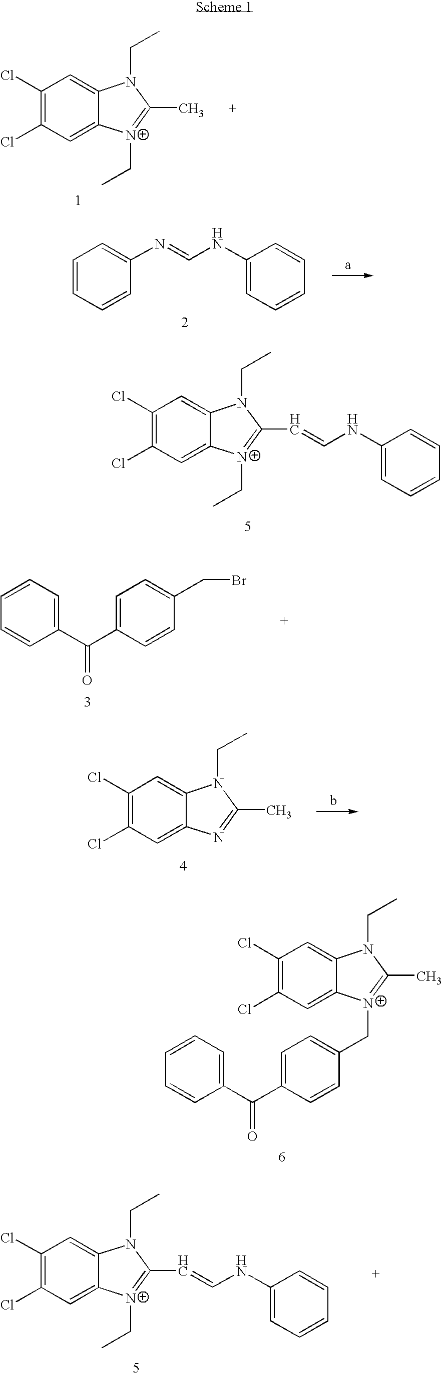

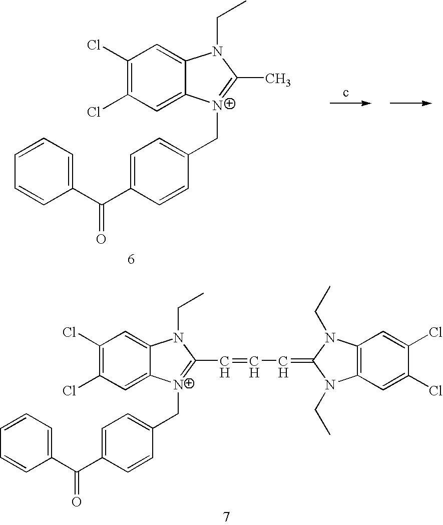

Activation of the known 5,6-dichloro-1,3-diethyl-2-methylbenzimidazolium salt 1 with N,N′-diphenylformamidine 2 in hot acetic anhydride affords 5 (reaction a). 6 is prepared from the reaction of 4-bromomethylbenzophenone 3 and 5,6-dichloro-1-ethyl-2-methylbenzimidazole 4 (reaction b). Condensation of 5 with 6 with acetic anhydride affords 7, a photolabeled carbocyanine dye, which contains the benzophenone photolabel in one of the side chains (reaction c).

c) ii) Methods of Attaching Reactive Groups to a Carbocyanine Dye

In Scheme 2, a general preparatory synthesis for a reactive version of compound (A) is presented.

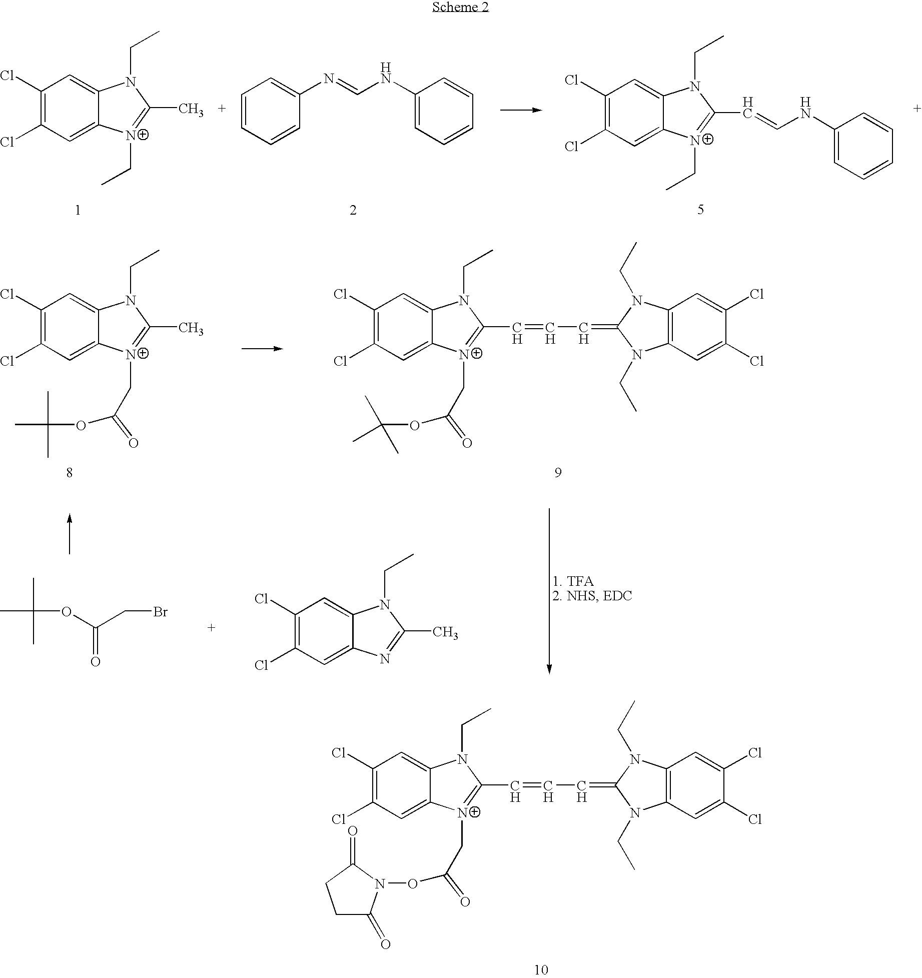

Activation of the known 5,6-dichloro-1,3-diethyl-2-methylbenzimidazolium salt (1) with N,N′-diphenylformamidine (2) in hot acetic anhydride afforded the novel intermediate 5. Condensation of 5 with the novel intermediate 8 (which was prepared from the t-butyl bromoacetate and 5,6-dichloro-1-ethyl-2-methylbenzimidazole) with acetic anhydride afforded 9. The t-butyl ester in 9 is removed by treatment with excess TFA, and the resulting carboxylate is converted into the amine-reactive ester 10.

Methods of Use

The present invention also provides methods of using the compounds described herein for a wide variety of chemical, biological and biochemical applications.

a) Detecting the Presence of Anionic and Non-Anionic Proteins

In one aspect, the present invention provides a method for detecting the presence of an anionic protein and the presence of a non-anionic protein in a sample. The method includes contacting the sample with at least one carbocyanine compound according to Formula I as described above. The product of this contacting is then incubated for a sufficient amount of time to allow the compound to associate with a protein selected from the anionic protein and the non-anionic protein. Then, the product of this step is illuminated with a first appropriate wavelength whereby the presence of said anionic protein in said sample is determined. Next, the product of this step is illuminated with a second appropriate wavelength whereby the presence of said non-anionic protein in said sample is determined.

Specifically the method comprises:
a) contacting the sample with a dye to form a labeled sample, wherein the dye has the following formula according to Formula I;
b) incubating the labeled sample for sufficient time to allow the dye to associate with an anionic protein and non-anionic protein to form an incubated sample;
c) illuminating the incubated sample with a first appropriate wavelength for observing the anionic proteins to form a first illuminated sample;
d) illuminating the incubated sample with a second appropriate wavelength for observing non-anionic proteins to form a second illuminated sample;
e) observing the first and the second illuminated sample whereby the presence of anionic and non-anionic proteins are determined.

In an exemplary embodiment the method includes contacting the sample with at least one of compound according to Formula I wherein Y¹ and Y² are N as described above. In another exemplary embodiment the method includes contacting the sample with at least one compound according to Formula II as described above. In another exemplary embodiment the method includes contacting the sample with Compound A as described above. In another exemplary embodiment, the compound further comprises a reactive group which is a member selected from an acrylamide, an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine, a hydrazide, an imido ester, an isocyanate, an isothiocyanate, a maleimide, a phosphoramidite, a reactive platinum complex, a sulfonyl halide, a thiol group, and a photoactivatable group. In another exemplary embodiment, the compound further comprises a carrier molecule which is a member selected from an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus, and combinations thereof.

In another exemplary embodiment, the sample is in a cuvette. In yet another embodiment, the sample is immobilized on a solid or semi solid support. In one aspect, the solid or semi-solid support is, but is not limited to, a polymeric microparticle, polymeric membrane, polymeric gel or glass slide.

In another exemplary embodiment, prior to contacting the sample with the compounds of the invention, the sample is immobilized on a gel and electrophoretically separated on the gel. This separation allows resolution of individual components of the sample and assignment of anionic properties to the individual components based upon differential dye staining.

In an exemplary embodiment, the anionic proteins are phosphoproteins, calcium binding proteins, sulfoproteins, or sialoglycoproteins.

Detecting the Presence of Anionic Proteins

In another aspect, the present invention provides a method for detecting the presence of an anionic protein in a sample. The method includes contacting the sample with at least one compound according to Formula I as described above. The product of this contacting is then incubated for a sufficient amount of time in order to allow the compound to associate with the anionic protein. Then, the product of this step is illuminated with a first appropriate wavelength whereby the presence of said anionic protein in said sample is determined.

In an exemplary embodiment the method includes contacting the sample with at least one compound according to Formula II of Compounds B-H above. In another exemplary embodiment the method includes contacting the sample with Compound A as described above.

In a specific embodiment, the method comprises:
a) contacting the sample with a dye to form a labeled sample, wherein the dye has the following formula according to Formula I, Formula II or Compounds B-H;
b) incubating the labeled sample for a sufficient amount of time to allow the dye to associate with the anionic proteins to form an incubated sample;
c) illuminating the incubated sample with an appropriate wavelength to form an illuminated sample;
d) observing the illuminated sample whereby the presence or absence of the anionic proteins is determined

Both of the methods described above can be used without limitation for the analysis and monitoring of anionic proteins and, optionally, non-anionic proteins (referred to herein as “targets”). In this way, targets can be detected in unlimited assay formats that provide information about the number of anionic groups on the target molecule, the identification of enzymes involved in addition or removal of these anionic groups, the role that such targets have in the proteome and—with further analysis—the site of attachment of anionic groups on the targets. Further analysis can be carried out after the compounds of the present invention are used to selectively detect and/or isolate the targets.

The methods of the present invention can be carried out on samples that are immobilized, on samples in which the carbocyanine dye is immobilized or where both the sample and carbocyanine dye are in solution. When the sample is immobilized on a solid or semi-solid support, the carbocyanine dye is typically incubated with the sample under conditions that maximize contact, such as gentle mixing or rocking.

The methods of the present invention for detecting targets that have been immobilized on a gel comprise the following steps:
i) immobilizing the sample on a gel;
ii) optionally contacting the gel of step i) with a fixing solution;
iii) contacting the gel of step ii) with a carbocyanine dye of the present invention
iv) incubating the gel of step iii) and the carbocyanine dye for sufficient time to allow said carbocyanine dye to associate with said target;
v) washing away excess dye
vi) illuminating the sample and/or the first target with a first appropriate wavelength, whereby the presence of the first target, such as an anionic protein, is detected; and,
vii) optionally illuminating the sample and/or the second target with a second appropriate wavelength, whereby the presence of the second target, such as a non-anionic protein is detected; and,
viii) optionally, a second (or third) stain is added to the gel to detect either total protein or proteins of another class, such as glycoproteins, or both.
ix) illuminating the sample and/or the target with an appropriate wavelength, whereby the presence of the target of the second or third stain is detected.

Typically, immobilizing the sample on a gel comprises electrophoretically separating the sample. The gel, without limit, includes any gel known to one of skill in the art for separating targets from each other, including polymer-based gels such as agarose and polyacrylamide wherein an electrical current is passed through the gel and the target molecules migrate based on charge and size. Thus, gels (reduced and native) also include both one and two-dimensional gels, and isoelectric focusing gels. Capillary electrophoresis may be employed using gels, solutions containing polymers, or solutions alone.

The staining solution can be prepared in a variety of ways, which is dependent on the medium the sample is in. A particularly preferred staining solution is one that is formulated for detection of anionic proteins in a gel. Specifically, the staining solution comprises a fluorescent carbocyanine compound of the present invention in an aqueous solution; optionally the staining solution comprises an organic solvent and a buffering component. The selection of the fluorescent carbocyanine compound dictates, in part, the other components of the staining solution. Any of the components of the staining solution can be added together or separately and in no particular order wherein the resulting staining solution is added to the gel. Alternatively, the components of the staining solution can be added to a gel in a step-wise fashion. The fluorescent compound is prepared by dissolving in a solvent, such as water, dimethylsulfoxide (DMSO), dimethylformamide (DMF), methanol, ethanol or acetonitrile, usually at a final concentration of about 0.1 μM to 100 μM, preferably the fluorescent carbocyanine compound is present in the staining solution at a concentration of about 0.5 μM to 20 μM.

The present staining formulation is typically neutral or slightly basic, which can be modified by the inclusion of a buffer. Useful buffering agents include salts of formate, acetate, 2-(N-morphilino)ethanesulfonic acid, imidazole, N-2-hydroxyethyl-piperazine-N′-2-ethanesulfonic acid (PIPES), Tris(hydroxymethyl)aminomethane acetate, or Tris (hydroxymethyl)aminomethane hydrochloride, 3-(N-morpholino)propanesulfonic acid (MOPS). The family of Good's buffers, including TRIS, MES, PIPES, MOPS, are preferred for the present methods. An exemplified buffering agent is MOPS. The buffering agent is typically present in the staining mixture at a concentration of about 1 mM to 300 mM; preferably the concentration is about 20 mM to 100 mM.

Inclusion of a water-miscible organic solvent, typically an alcohol, in the staining solution is recommended when the staining solution contains a pH-buffering agent and a salt. Although the use of highly polar solvents such as formamide is permitted, typically, the polar organic solvent is an alcohol having 1-6 carbon atoms, or a diol or triol having 2-6 carbon atoms. A preferred alcohol is ethanol. The polar organic solvent, when present, is typically included in the staining solution at a concentration of 5-50%. The presence of a polar organic solvent is particularly advantageous when staining sodium dodecyl sulfate (SDS)-coated proteins, as is typically the case when staining phosphorylated proteins or peptides that have been electroblotted from SDS-polyacrylamide gels. Typically, in the preferred procedure, SDS is removed from a gel or blot prior to addition of the binding solution by fixing and washing; however, some SDS may remain and can interfere with the binding methods of the present invention. Without wishing to be bound by any theory, it appears that the presence of an alcohol improves luminescent labeling of phosphorylated proteins or peptides by removing any SDS that was not removed by washing or fixing the sample. However, nitrocellulose membranes may be damaged by high concentrations of alcohol (for example, greater than about 20%), and so care should be taken to select solvent concentrations that do not damage the membranes upon which the phosphorylated proteins or peptides are immobilized.

In an exemplary embodiment, a staining solution comprise 4 μM of a present carbocyanine dye, 10% ethanol, and 20 mM MOPS at pH 7.25. See, Example 1.

Optionally, a sample separated on a gel may be transferred to a polymeric membrane, using techniques well known to one skilled in the art, wherein the membrane is then contacted with a carbocyanine dye of the present invention to selectively detect the targets. A method of the present invention for detecting the targets immobilized on a membrane comprises the following steps:
i) electrophoretically separating the sample on a gel;
ii) transferring the separated sample to a membrane;
iii) optionally contacting the membrane of step ii) with a fixing solution;
iv) contacting the membrane of step iii) with a carbocyanine dye of the invention;
v) incubating the membrane of step iv) and the a carbocyanine dye for sufficient time to allow the compound to associate with the targets; and,
vi) washing away excess dye;
vii) illuminating the sample and/or the first target with a first appropriate wavelength, whereby the presence of the first target, such as an anionic protein, is detected; and,
viii) optionally illuminating the sample and/or the second target with a second appropriate wavelength, whereby the presence of the second target, such as a non-anionic protein is detected; and,
ix) optionally, a second (or third) stain is added to the gel to detect either total protein or proteins of another class, such as glycoproteins, or both.
x) illuminating the sample and/or the target with an appropriate wavelength, whereby the presence of the target of the second or third stain is detected.

Protein gel electrophoresis is typically performed using SDS as a component of either the sample preparation or in the running buffer. However, SDS interferes with the carbocyanine dyes of the invention and therefore must be removed from the gel or membrane prior to addition of the binding solution. Gels and membranes are fixed and washed, which results in the removal of most or all of the SDS from the gels or blots. A preferred fixing solution for gels and membranes comprises methanol and acetic acid; optionally the fixing solution comprises glutaraldehyde. The methanol is present at a concentration of about 35-50% and the acetic acid is present at about 0-15% and the glutaraldehyde is present at about 0-2%. Typically, washing the gels or membranes with 100% water follows fixing.

However, for purposes of the invention, the carbocyanine dyes of the invention also detect targets that have been separated on a native or non-reduced gel. Therefore, for methods utilizing these gels that do not contain SDS, the fixing solution step is not necessary.

After samples have been separated on a gel or transferred to a polymeric membrane, optionally fixed, and washed, the gel or blot is incubated with a carbocyanine dye of the invention (Examples 1-2). The targets are incubated with the carbocyanine dye for a time sufficient for the dye to bind to the targets that are present. Preferably, this time is not more than 24 hours, more preferably this time is less than 8 hours and most preferably this incubation time is less than 2 hours, but not less than 5 minutes. After incubation with the carbocyanine dye the gels or membranes are typically washed with a mixture that preferably comprise an acidic buffering agent and acetonitrile; useful buffering agents to be used with the present invention include, without limitation, NaOAc, formate and 2-(N-morpholino)ethanesulfonic acid. Typically, the buffering agent is present in the washing solution at a concentration of about 25 mM to about 100 mM. In addition, it has been found that optional inclusion of acetonitrile in the washing solution usually reduces non-specific labeling. Preferably, acetonitrile is present at a concentration from 1-7%, more preferably 3-4%. An alternative washing solution is comprised of 10-20% 1,2-propanediol.

Thus, following binding of the carbocyanine dye with the target and washing, the carbocyanine dye-target complex can be illuminated directly so as to visualize the location, quantity, or presence of the targets.

A particular advantage to identifying targets in a 2-D gel is the ability to correctly identify the target, as well as to quantitate post-translational modification of proteins for the addition or subtraction of anionic groups. Specifically, labeling of anionic proteins while doing concurrent, or subsequent, total protein staining identifies the anionic proteome, while the intensity of the signal can be correlated to the level of anionic protein, when compared to the total protein stain. Any fluorescent dye specific for total proteins can be used to stain total proteins in the gel; a preferred stain is SYPRO® Ruby dye for gels or any dye disclosed in U.S. Pat. No. 6,316,276. Other fluorescent dyes such as MDPF and CBQCA could also be used for detection on membranes. Because SDS is removed by washing prior to staining with the staining mixture of the present invention, total protein stains such as SYPRO® Ruby dye are preferred because SDS is not critical for their staining function. However, protocol changes can be made when using a stain that requires SDS for staining sensitivity, such as SYPRO® Orange dye, SYPRO® Red dye and SYPRO® Tangerine dye, by adding SDS back to the gel prior to a total protein stain step and including SDS in the staining solution for the total protein stain (Malone et al. Electrophoresis (2001) 22(5):919-932). A preferred mixture for returning SDS back to a gel is 2% acid/0.0005% SDS, and optionally 40% ethanol, wherein the gel is incubated for at least one hour. Alternatively, the total protein stain can be performed prior to the anionic target molecules staining of the present invention; therefore, in this case, it is not necessary to add back the SDS to the gel, but simply to remove the SDS prior to the anionic target molecule staining step, as contemplated by the present invention. Therefore, alternative preferable total protein stains for gels include but are not limited to, SYPRO® Orange dye, SYPRO® Tangerine dye and SYPRO® Red dye or any dye disclosed in U.S. Pat. Nos. 5,616,502 and 6,579,718. Alternative, but less preferred, total protein stains for gels include Coomassie Blue or silver staining, which utilize staining techniques well known to those skilled in the art. Alternative total proteins stains useful for staining blots are SYPRO® Rose Plus dye and DyeChrome™ dye or any dye solution disclosed in U.S. Pat. No. 6,329,205 and U.S. Ser. No. 10/005,050.

Another very important advantage when labeling anionic proteins in a 2-D gel is to include a stain for glycoproteins, wherein a 3-way analysis of the proteome could be accomplished (Steinberg et al., “Rapid and Simple Single Nanogram Detection of Glycoproteins in Polyacrylamide Gels and on Electroblots,” Proteomics 1:841-855 (2001)). A preferred glycoprotein stain is Pro-Q® Emerald 300 dye or Pro-Q® Emerald 488 dye or any other dye disclosed in U.S. Ser. No. 09/970,215. In addition, if the sample comprises fusion proteins with oligohistidine affinity peptides, Pro-Q® Sapphire 365, 488, or 532 dye or InVision stain (Invitrogen Corp.) can be used to simultaneously detect these proteins or peptides.

Thus, it is particularly advantageous that the parallel determination of both protein expression levels and functional attributes of the anionic proteins can be achieved with the present invention within a single 2-D gel electrophoresis experiment. Analysis can be accomplished by using image analysis software, e.g., Compugen's Z3 program or Phoretix Progenesis software. Any two images can be re-displayed, allowing visual inspection of the differences between the images, and quantitative information can be readily retrieved in tabular form with differential expression data calculated.

Alternatively, single-dimension polyacrylamide and corresponding blots can be simultaneously or subsequently stained for total proteins or glycoproteins using staining techniques and dyes described above. A particular advantage for counterstaining a gel or blot that has been labeled using methods of the present invention is the ability to distinguish between nonspecific labeling and labeling of anionic proteins with a low number of anionic groups. This is important for accurately identifying anionic molecules that have undergone a small change in the degree of phosphorylation, for example. Counterstaining a blot or gel with a total protein stain such as SYPRO® Ruby dye permits a ratiometric analysis of the fluorescent signal generated from the carbocyanine dyes of the present invention compared to the fluorescent signal generated from a total protein stain. This ratiometric analysis also permits the stoichiometry determination of the anionic proteins relating to, for example, the overall phosphorylation state of the molecule as well as the addition or subtraction of phosphate groups.

Another particular advantage for staining anionic proteins separated in polyacrylamide gels is for the analysis of proteins of interest by combining spot detection with the compounds of this invention with mass spectrometry techniques for further analysis. For example, because anionic proteins may co-migrate in a gel, further analysis may be essential or desired to specifically identify and analyze the anionic protein of interest. This further analysis can be achieved by measurement of a set of peptide masses derived from a protein, i.e., by peptide mapping with mass spectrometry (MS), or by obtaining amino acid sequence information from individual peptides, i.e., protein sequencing by MS/MS or by Edman degradation. Thus, a protein band or spot, once identified using the compositions and methods of the present invention, may be excised from the gel, rinsed, optionally reduced and S-alkylated, and then digested in situ in the gel with a sequence-specific protease, e.g., trypsin, using standard protocols. See Shevchenko et al., “Mass Spectrometric Sequencing of Proteins from Silver Stained Polyacrylamide Gels,” Anal. Chem. 68:850-58 (1996). The peptide mixture thus generated may be extracted from the gel and analyzed by MS, using standard protocols. Peptide mapping by matrix-assisted laser desorption/ionization (MALDI) mass spectrometry is often most sensitive. Methods for the in-gel digestion of proteins are described in Jensen et al., “Mass Spectrometric Identification and Microcharacterization of Proteins From Electrophoretic Gels: Strategies and Applications,” PROTEINS: Structure, Function, and Genetics Suppl. 2:74-89 (1998).

Through the addition of certain molecules to the sample, the carbocyanine dyes of the invention are able to further differentiate between proteins and non-proteins, anionic and non-anionic proteins, and finally, between particular classes of anionic proteins.

Through the addition of certain molecules to the sample, the selectivity of carbocyanine dyes of the invention for anionic proteins over non-anionic proteins can be improved. Thus, in an exemplary embodiment, the carboxylic acid moieties of the proteins in a sample can be converted to ester moieties. In this way, binding of the carbocyanine dyes to the peptide backbones of proteins can be minimized, improving selective detection of phosphoproteins or sulfated glycoproteins.

Through the addition of certain molecules to the sample, the selectivity of carbocyanine dyes of the invention for certain classes of anionic proteins over other classes of anionic proteins can be improved. Thus, in an exemplary embodiment, calcium is added to the sample prior to the contacting of the sample with the carbocyanine dyes of the invention. In this way, reduced binding of the carbocyanine dye to the calcium-binding pocket of the calcium-binding proteins of the sample is achieved. This allows for the preferential detection of phosphoproteins, sulfoproteins, and sialoglycoproteins in the sample. In another exemplary embodiment, a phosphatase is added to the sample prior to the contacting of the sample with the carbocyanine dyes of the invention. In this way, the phosphate groups on the phosphoproteins of the sample will be removed. This allows for the preferential detection of calcium-binding proteins, sulfoproteins, and sialoglycoproteins in the sample. In yet another exemplary embodiment, a sulfatase is added to the sample prior to the contacting of the sample with the carbocyanine dyes of the invention. In this way, the sulfate groups on the sulfoproteins of the sample will be removed. This allows for the preferential detection of calcium-binding proteins, phosphoproteins, and sialoglycoproteins in the sample. sulfoproteins. In yet another exemplary embodiment, a neuramidinase is added to the sample prior to the contacting of the sample with the carbocyanine dyes of the invention. In this way, the sialo groups on the sialoglycoproteins of the sample will be removed. This allows for the preferential detection of calcium-binding proteins, phosphoproteins, and sulfoproteins in the sample.

Sample Illumination

The sample is illuminated with a wavelength of light selected to give a detectable optical response, and observed with a means for detecting the optical response. Equipment that is useful for illuminating the present compounds and compositions of the invention includes, but is not limited to, hand-held ultraviolet lamps, mercury arc lamps, xenon lamps, lasers and laser diodes. These illumination sources are optically integrated into laser scanners, fluorescence microplate readers or standard or microfluorometers.

The carbocyanine dyes of the invention may, at any time after or during an assay, be illuminated with a wavelength of light that results in a detectable optical response, and observed with a means for detecting the optical response. Upon illumination, such as by an ultraviolet or visible wavelength emission lamp, an arc lamp, a laser, or even sunlight or ordinary room light, the fluorescent compounds, including those bound to the complementary specific binding pair member, display intense visible absorption as well as fluorescence emission. Selected equipment that is useful for illuminating the fluorescent compounds of the invention includes, but is not limited to, hand-held ultraviolet lamps, mercury arc lamps, xenon lamps, argon lasers, laser diodes, and YAG lasers. These illumination sources are optionally integrated into laser scanners, fluorescence microplate readers, standard or mini fluorometers, or chromatographic detectors. This fluorescence emission is optionally detected by visual inspection, or by use of any of the following devices: CCD cameras, video cameras, photographic film, laser scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, a fluorescence microscope or a fluorometer, the instrument is optionally used to distinguish and discriminate between the fluorescent compounds of the invention and a second fluorophore with detectably different optical properties, typically by distinguishing the fluorescence response of the fluorescent compounds of the invention from that of the second fluorophore. Where a sample is examined using a flow cytometer, examination of the sample optionally includes isolation of particles within the sample based on the fluorescence response by using a sorting device.

In another embodiment, the illumination source is used to form a covalent bond between the present carbocyanine dye and the anionic protein. In this instance the carbocyanine dye comprises a photoactivatable reactive group, such as those disclosed above.

Sample Preparation

The end user will determine the choice of the sample and the way in which the sample is prepared. The sample includes, without limitation, any biologically derived material that is thought to contain an anionic protein or non-anionic protein. Alternatively, samples also include material in which an anionic protein has been added.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, β cells, hepatocytes, and neurons.

In many instances, it may be advantageous to add a small amount of a non-ionic detergent to the sample. Generally the detergent will be present in from about 0.01 to 0.1 vol. %. Illustrative non-ionic detergents include the polyoxyalkylene diols, e.g. Pluronics, Tweens, Triton X-100, etc.

In fluorescence experiments, the reaction is optionally quenched. Various quenching agents may be used, both physical and chemical. Conveniently, a small amount of a water-soluble solvent may be added, such as acetonitrile, DMSO, SDS, methanol, DMF, etc.

Kits

In another aspect, the present invention provides kits that include a carbocyanine dye of the invention. The kit will generally also include instructions that teaches a method of the invention and/or describes the use of the components of the kit.

Thus, in an exemplary embodiment, the compound has a formula selected from: 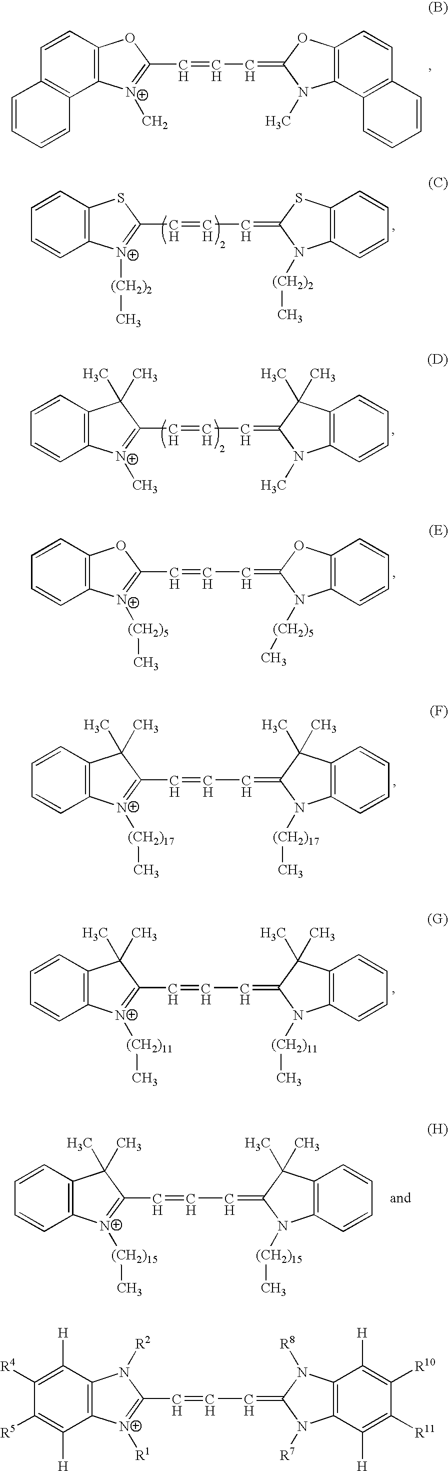
in which R¹, R², R⁷, and R⁸ are substituted or unsubstituted alkyl; and R⁴, R⁵, R¹⁰, and R¹¹ are halogen.

In another exemplary embodiment, the compound is

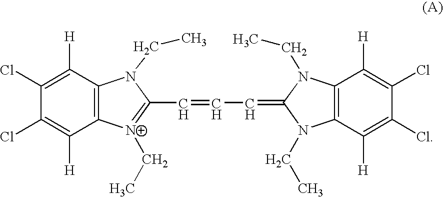

Any sample that is suspected of containing anionic proteins can be analyzed by the kits of the present invention. The sample is typically an aqueous solution such as a body fluid from a host, for example, urine, whole blood, plasma, serum, saliva, semen, stool, sputum, cerebral spinal fluid, tears, mucus or the like. In an exemplary embodiment, the sample is plasma or serum. The sample can be pretreated if desired and can be prepared in any convenient medium that does not interfere with the assay. For example, the sample can be provided in a buffered synthetic matrix.

The sample suspected of containing the anionic protein and a calibration material containing a known concentration of the anionic protein are assayed under similar conditions. Anionic protein concentration is then calculated by comparing the results obtained for the unknown specimen with results obtained for the standard. This is commonly done by constructing a calibration or dose response curve.

Various ancillary materials will frequently be employed in an assay in accordance with the present invention. In an exemplary embodiment, buffers and/or stabilizers are present in the kit components. In another exemplary embodiment, the kits comprise indicator solutions or indicator “dipsticks”, blotters, culture media, cuvettes, and the like. In yet another exemplary embodiment, the kits comprise indicator cartridges (where a kit component is bound to a solid support) for use in an automated detector. In another exemplary embodiment, the kit further comprises molecular weight markers, wherein said markers are selected from phosphorylated and non-phosphorylated polypeptides, calcium-binding and non-calcium binding polypeptides, sulfonated and non-sulfonated polypeptides, and sialylated and non-sialylated polypeptides. In another exemplary embodiment, the kit further comprises a member selected from a fixing solution, a detection reagent, a standard, a wash solution, and combinations thereof. In still another exemplary embodiment, additional proteins, such as albumin, or surfactants, particularly non-ionic surfactants, may be included. In still another exemplary embodiment, the detection reagent in the kit is a compound that associates with all proteins, a compound that preferentially associates with cationic proteins, a compound which preferentially associates with glycoproteins, and an antibody.

A detailed description of the invention having been provided above, the following examples are given for the purpose of illustrating the invention and shall not be construed as being a limitation on the scope of the invention or claims.

### EXAMPLES

### Example 1

Serial Dichromatic Detection of Proteins in SDS-Polyacrylamide Gels Using (A) and SYPRO Ruby Protein Gel Stain

Proteins were separated by SDS-polyacrylamide gel electrophoresis utilizing 13% T, 2.6% C gels. % T is the total monomer concentration expressed in grams per 100 mL and % C is the percentage crosslinker. The 0.75 mm thick, 6×10 cm gels were subjected to electrophoresis using the Bio-Rad mini-Protean III system according to standard procedures. Following separation of the proteins on SDS-polyacrylamide gels, the gels were fixed for one hour in 100 mL of 50% ethanol/7% acetic acid and then fixed overnight in 100 mL of fresh fixative solution to ensure complete elimination of SDS. Gels were next washed 3 times for 20 min each in deionized water. The gels were then incubated in a staining solution containing 4 μM (A), 10% ethanol, 20 mM MOPS at pH 7.25 for 2 h in a total volume of 50 mL. Afterwards, the gels were washed for 30 min in 50 mL of 20% acetonitrile, 10 mM MOPS at pH 7.25. Finally, the gels were rinsed 30-60 min in deionized water. All incubation and wash steps were performed with gentle orbital shaking, typically at 50 rpm. Stained gels were protected from bright light exposure by covering with aluminum foil. The resulting red-fluorescent signal produced by the (A) J-aggregate form was visualized using the 488 nm excitation line of the argon ion laser on the FX Pro Plus imager (Bio-Rad Laboratories, Hercules Calif.) with a 640 nm band-pass emission filter. The green fluorescent signal produced by the (A) dye monomer form could be visualized using the 488 nm excitation line of the argon ion laser on the FX Pro Plus imager and a 530 nm band-pass emission filter.

Following selective staining of proteins with (A), the gels were incubated overnight in 80 mL of SYPRO Ruby protein gel stain in order to detect the total protein profile. The gels were incubated in 50 mL 7% acetic acid, 10% methanol for 30 min and then washed with deionized water for 30 min. The resulting red fluorescent signal was visualized using the 488 nm excitation line of the argon ion laser on the FX Pro Plus imager (Bio-Rad Laboratories, Hercules Calif.) with a 555 nm long-pass emission filter, or with the 473 nm excitation line of the SHG laser on the Fuji FLA-3000G Fluorescence Image Analyzer (Fuji Photo, Tokyo, Japan) with a 580 nm band pass emission filter.

### EXAMPLE 2

Selectivity of Staining of the Phosphoprotein Chicken Ovalbumin Relative to the Nonphosphorylated Protein Bovine Serum Albumin in SDS Polyacrylamide Gel Electrophoresis

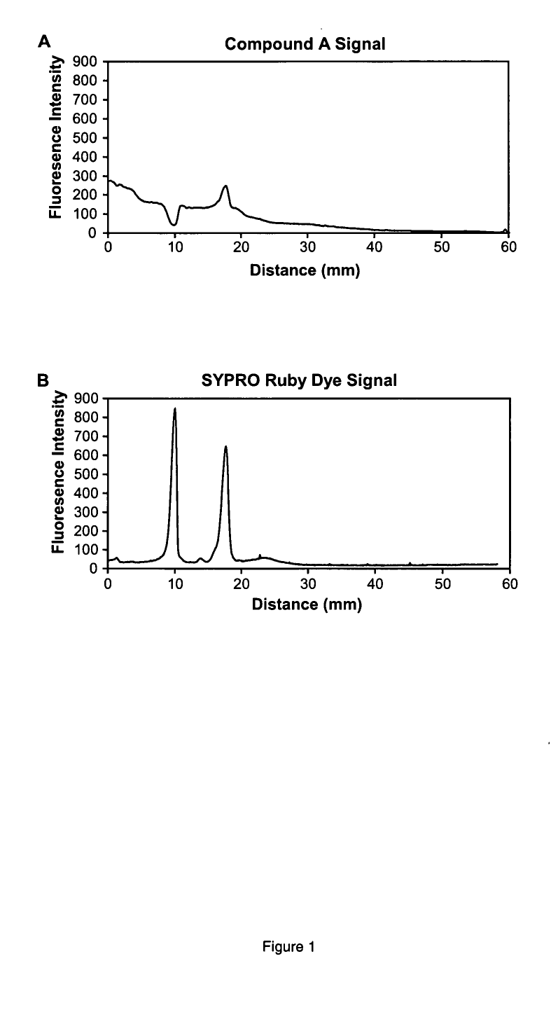

A mixture containing 500 ng each of the purified proteins chicken ovalbumin and bovine serum albumin was prepared in 1×SDS sample buffer (50 mM Tris, 10% glycerol, 50 mM DTT, 2% SDS, and 0.01% bromophenol blue, pH 6.8). Proteins were separated by SDS-polyacrylamide gel electrophoresis, stained with (A), imaged, stained for total proteins with SYPRO Ruby dye, and imaged again as described in Example 1. Bovine serum albumin was negatively stained by (A) aggregate, such that the signal was less than the background signal of (A) aggregate, obtained from a blank region of the gel. Chicken ovalbumin was positively stained by (A) aggregate, such that the signal was higher than the background signal of unbound (A) aggregate. Post-staining of the gel with SYPRO Ruby dye shows two peaks of approximately equal intensity; see FIG. 1.

### EXAMPLE 3

Selective Staining of Anionic Proteins in SDS Polyacrylamide Gel Electrophoresis

Samples containing 500 ng of pepsin and α casein (both phosphoproteins), chicken ovalbumin (a phosphorylated glycoprotein), Tamm-Horsfall glycoprotein, (contains numerous sulfated glycans), α₁ acid glycoprotein (an acidic glycoprotein), avidin (a basic glycoprotein), and the proteins phosphorylase b, bovine serum albumin, carbonic anhydrase, and lysozyme were prepared in 1×SDS sample buffer. Proteins were separated by SDS-polyacrylamide gel electrophoresis, stained with (A), imaged, stained for total proteins with SYPRO Ruby dye, and imaged again as described in Example 1. The (A) aggregate stained the acidic phosphoprotein, pepsin, the most strongly of the proteins in the sample, followed by α casein, at 79% of the intensity of pepsin. The (A) aggregate stained α₁ acid glycoprotein, Tamm-Horsfall glycoprotein, and chicken ovalbumin at 29%, 12%, and 3% relative to pepsin. Phosphorylase b, bovine serum albumin, carbonic anhydrase, lysozyme, and the basic phosphoprotein, avidin were all negatively stained by (A) aggregate, such that the signal was less than the background signal of (A) aggregate, obtained from a blank region of the gel.

### EXAMPLE 4

Selective Staining of Anionic Proteins in Polyacrylamide Gels that are First Separated by Microscale Solution-Phase Isoelectric Focusing

Extracts of bovine heart mitochondria were injected into a Zoom IEF Fractionator chamber (Invitrogen Corporation, Carlsbad, Calif.) and subjected to isoelectric focusing into five fractions having the pH ranges of pH 3.0-4.6, pH 4.6-5.4, pH 5.4-6.2, pH 6.2-7.0, and pH 7.0-10.0 according to the manufacturer's protocol. Samples of each fraction were separated by SDS-polyacrylamide gel electrophoresis, stained with (A), imaged, stained for total proteins with SYPRO Ruby dye, and imaged again as described in Example 1. A majority of proteins in the three fractions spanning the pH range of pH 3.0-6.2 were stained by the (A) aggregate. A minority of proteins in the pH 3.0-6.2 range were not stained by the (A) aggregate. No proteins in the two fractions spanning the pH range of pH 6.2-10.0 were stained by the (A) aggregate. The gel lanes containing the pH 6.2-7.0 and pH 7.0-10.0 fractions were negatively stained by the (A) aggregate, such that the signal was less than the background signal of (A) aggregate obtained from a blank lane of the gel.

### EXAMPLE 5

Selective Staining of Anionic Proteins in 2-D Gel Electrophoresis

For 2-D gel electrophoresis, whole cell lysates of Jurkat cells were prepared by standard procedures. Proteins were quantified by the Bicinchoninic Acid (BCA) solution assay method using bovine serum albumin as the standard protein. All samples were precipitated before 2-D gel electrophoresis to minimize unspecific staining due to phospholipids and other cell constituents. Approximately 150 μg protein sample was applied to each pH 4-7 Immobiline Drystrip immobilized pH gradient gel (Amersham Life Sciences, Piscataway, N.J.)) by the in-gel rehydration method. All proteins were focused for 80,000 volt-hours using a pHaser IEF focusing unit (Genomic Solutions, Ann Arbor, Mich.). After completion, 12.5% large format SDS polyacrylamide gel electrophoresis was performed using an Investigator™ system (Genomic Solutions, Ann Arbor, Mich.). Gels were fixed overnight in 700 mL of fresh fixative solution to ensure complete elimination of SDS. Gels were next washed 3 times for 45 min each in deionized water. The gels were then incubated in 500 mL of staining solution containing 4 μM (A); 10% ethanol; 20 mM MOPS at pH 7.25 for 2 h. Afterwards, the gels were washed for 30 min in 500 mL of 10 mM MOPS at pH 7.25. All incubation and wash steps were performed with gentle orbital shaking, typically at 40 rpm. Stained gels were protected from bright light exposure by covering with aluminum foil. Following selective staining of proteins with (A), the gels were incubated overnight in 500 mL of SYPRO Ruby protein gel stain. The gels were then incubated in 500 mL 7% acetic acid, 10% methanol for 30 min and then washed with deionized water for 30 min. Gels were imaged as described in Example 1.

Two-color overlay images of the 2-D gels were generated using Z3 software (Compugen, Tel Aviv, Israel). This software package uses raw-image-based computation of registration, region-based matching, and a complementary pseudocolor visualization technique to highlight differences in 2-D gel profiles. With the system, spots of the reference gel appear green and those of the comparative gel appear magenta. When images are aligned, similarly expressed spots in the overlay image appear gray or black, while those that differ in expression levels appear green or magenta. This facilitates identification of differentially expressed protein spots by simple visual inspection. Differential display analysis of (A) compared with SYPRO Ruby protein gel stain (Molecular Probes, Eugene, Oreg.), a total protein stain, demonstrates that (A) aggregate selectively detects a subset of the proteome. It is particularly evident that certain anionic proteins are selectively visualized with the dye. One prominent protein detected with (A) is heat shock protein-90, a prominent phosphoprotein. A similar comparison of the staining profile of (A) with that of Pro-Q Diamond phosphoprotein gel stain (Molecular Probes, Eugene, Oreg.) shows that while some of the spots generated by the two stains coincide, others do not. This is undoubtedly due to the fact that (A) is selective for a range of anionic proteins that include sulfated proteins, phosphorylated proteins and calcium-binding proteins. However, it should be noted that some proteins identified as being phosphorylated by Pro-Q Diamond dye are not detected with (A).

### EXAMPLE 6

Selective Detection of Phosphoproteins Relative to Nonphosphoproteins in Solution

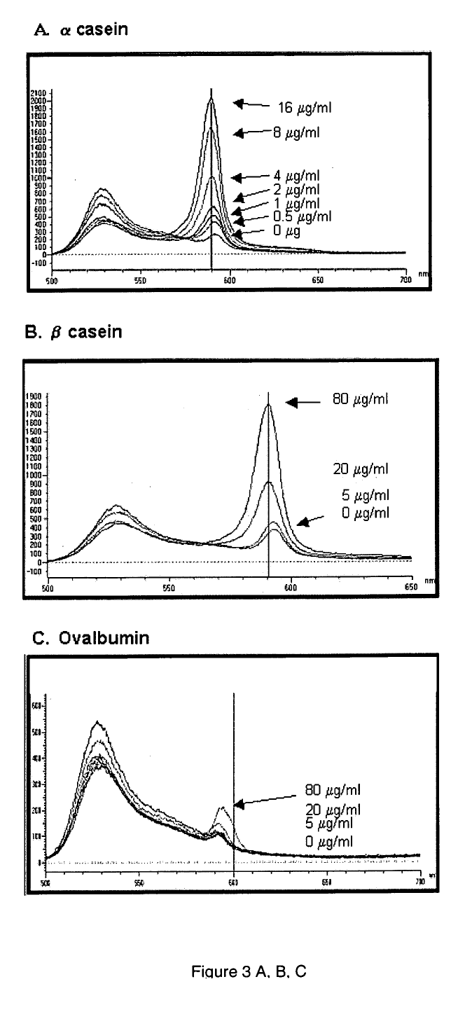

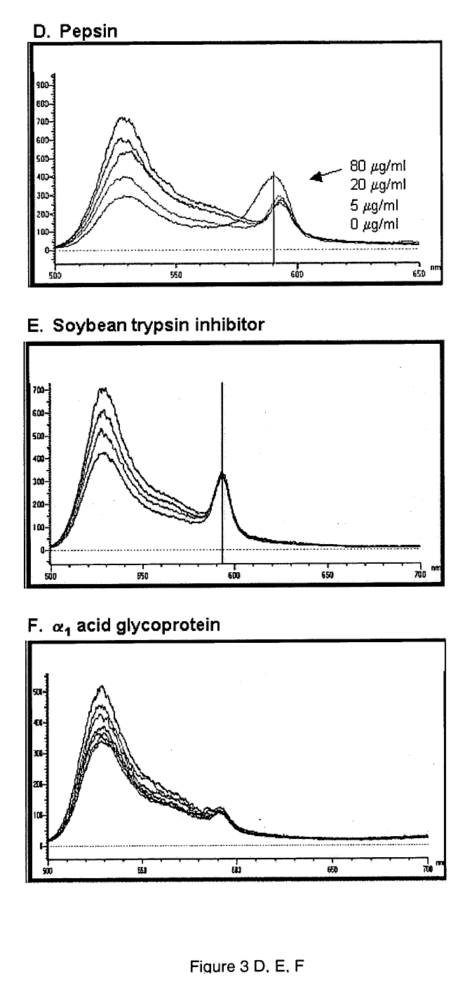

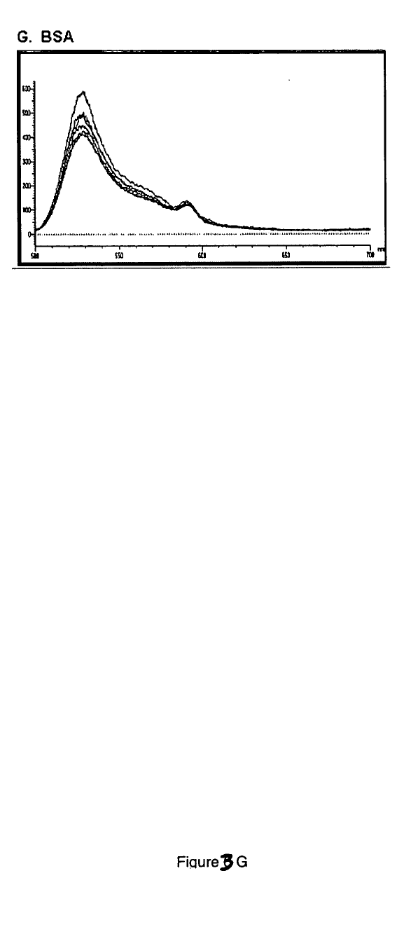

A solution was prepared containing 1 μM (A) in 10 mM MOPS pH 7.5 and 30% ethylene glycol. Emission spectra from 500 to 700 nm were obtained on a Hitachi F-4500 fluorescence spectrophotometer (Hitachi Instruments Inc., Tokyo, Japan) using an excitation wavelength of 488 nm. (A) in solution gave a large monomer peak at 532 nm and a small J aggregate peak at 594 nm. To this solution an increasing amount of each purified protein was titrated in, the solution was inverted to mix, and the emission spectra was immediately obtained. Titration spectra were obtained for the phosphoproteins β casein, at 0.5-16 μg/mL final concentrations, and α casein, ovalbumin, and pepsin, at 5-80 μg/mL final concentrations, and the nonphosphoproteins soybean trypsin inhibitor, α₁ acid glycoprotein, and bovine serum albumin, at 5-80 μg/mL final concentrations. For the four phosphoproteins the (A) aggregate peak intensity increased with increasing protein concentration, with a sensitivity of 0.5 μg/mL for β casein, 5 μg/mL for α casein, and 20 μg/mL for ovalbumin and pepsin. For the three nonphosphorylated proteins, the (A) aggregate peak did not increase over the full titration range. For all seven phosphorylated and nonphosphorylated proteins, the (A) monomer peak decreased with increasing protein concentration. See, FIG. 3

### Example 7

Limits of Detection of Phosphoproteins in SDS-Polyacrylamide Gels Using (A) Compared to Stains-All.

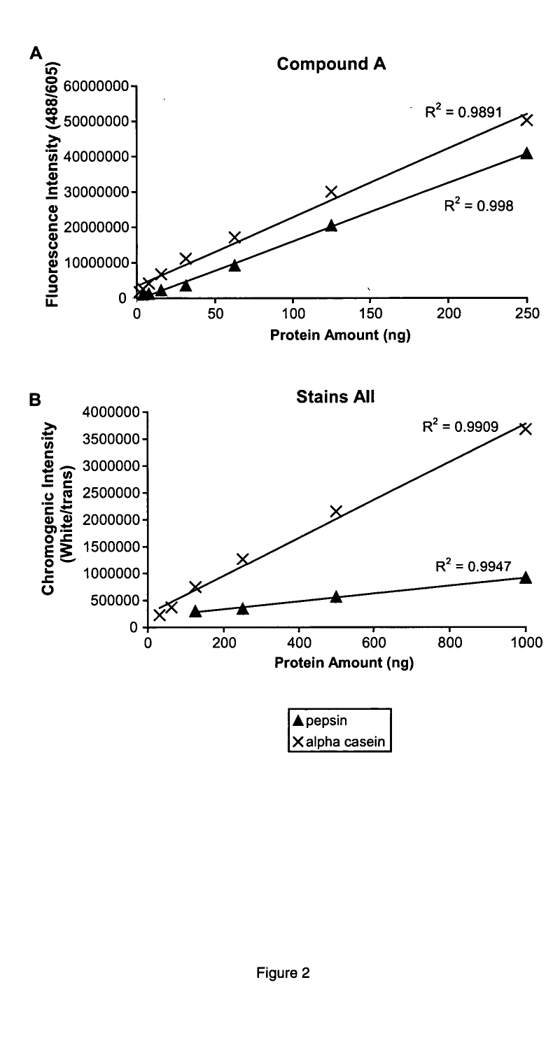

A two-fold dilution series of pepsin and of α casein, from 2000-0.5 ng, were prepared in 1×SDS sample buffer. Proteins were separated by SDS-polyacrylamide gel electrophoresis and stained as described in Example 1, except that one set of gels was incubated in a staining solution containing 20 μM Stains-All, 10% ethanol, 20 mM MOPS at pH 7.25 in a total volume of 50 mL. The resulting red-fluorescent signal produced by the (A) J-aggregate form was visualized using the 488 nm excitation line of the argon ion laser on the FX Pro Plus imager with a 605 nm band-pass emission filter. The resulting blue-purple chromogenic signal produced by Stains-All was visualized using white light trans illumination, with an 8.5 second exposure, and an F-stop of 11 on the FluorS Max imager (Bio-Rad Laboratories, Hercules Calif.). Limits of detection were determined to be 3.9 ng for pepsin and 0.5 ng for α casein using (A) and 125 ng for pepsin and 31.3 ng for α casein using Stains-All. Thus (A) was determined to be roughly 30-60 times more sensitive than Stains-All for the two proteins evaluated. See, FIGS. 2A and B.

### Example 8

### Preparation of Compound 7

A mixture of 5,6-dichloro-1,3-diethyl-2-methylbenzimidazolium iodide (3.00 g, 7.79 mmol) and N,N′-diphenylformamidine (1.53 g, 7.79 mmol) in 4 mL acetic anhydride was heated to 145° C. for 16 hours, then cooled and treated with dichloromethane (20 mL) and ether (20 mL). After stirring for 30 minutes, the resulting precipitate was collected by suction filtration, rinsed with ether, and dried in vacuo to give compound 5 as 2.63 g pale brown powder: TLC R_{f} 0.20 (chloroform:methanol:acetic acid 20:4:1); LCMS m/z 488 (488 calcd for C₁₉H₁₇N₃Cl₂). A solution of 1-ethyl-2-methyl-5,6-dichlorobenzimidazole (0.27 g, 1.2 mmol) and 4-bromomethylbenzophenone (0.36 g, 1.3 mmol) in 10 mL anhydrous acetonitrile was heated to reflux under a calcium sulfate drying tube and a condensor for 72 hours, then cooled to rt. The resulting precipitate was collected by suction filtration and dried in vacuo to give compound 6 as 0.40 g colorless powder: TLC R_{f} 0.05 (chloroform:methanol:acetic acid 50:5:1).

To a light brown mixture of compound 5 (0.24 g, 0.50 mmol) and compound 6 (0.25 g., 0.50 mmol) in 4 mL anhydrous DMF was added diisopropylethylamine (0.17 mL, 1.0 mmol) and acetic anhydride (0.31 g, 3.0 mL). The resulting brown mixture was stirred in darkness for 16 hours, then poured into 50 mL ethyl ether. The resulting precipitate was collected, rinsed with ether, an ddried in vacuo to give compound 7 as 0.36 g red powder: TLC R_{f} 0.45 (chloroform:methanol 85:15). Further purification was effected by flash chromatography on silica gel using methanol in chloroform as eluant, giving pure 7 as 63 mg of a red powder: LCMS m/z 676 (675.5 calcd for C₃₇H₃₃N₄Cl₄).

### Example 9

### Preparation of Compound 10

A solution of 1-ethyl-2-methyl-5,6-dichlorobenzimidazole (0.50 g, 2.2 mmol) and t-butyl bromoacetate (0.39 mL, 2.6 mmol) in 15 mL anhydrous acetonitrile was heated to reflux under a calcium sulfate drying tube and a condensor for 48 hours, then cooled to rt. The resulting precipitate was collected by suction filtration and dried in vacuo to give compound 8 as 0.75 g colorless powder: TLC R_{f} 0.05 (chloroform:methanol:acetic acid 50:5:1).

A brown mixture of compound 8 (0.75 g, 1.8 mmol) and compound 5 (0.86 g, 1.8 mmol)) in 15 mL anhydrous DMF was treated with diisopropylethylamine (0.61 mL, 3.5 mmol) and acetic anhydride (1.1 g, 11 mmol). The resulting mixture was stirred 48 hours, then poured into 150 mL ether. The resulging precipitate was collected by suction filtration, rinsed with ether, and dried in vacuo to give compound 9 as 1.4 g brown powder: TLC R_{f} 0.20 (chloroform:methanol 85:15). Further purification is effected by flash chromatography on silica gel to give pure compound 9 as a red powder: LCMS m/z 612 (611 calcd for C₂₉H₃₃N₄O₂Cl₄).

A solution of compound 9 (0.50 g) in 10 mL dichloromethane is treated with trifluoroacetic acid (TFA, 10 mL). The resulting solution is stirred for 4 hours, then concentrated in vacuo. Toluene (2×10 mL) is stripped from the residue to remove residual TFA. The resulting residue is dissolved in 10 mL anhydrous DMF and treated with 1.2 molar equivalents of EDC, then 1.1 molar equivalents of N-hydroxysuccinimide. The resulting solution is stirred at rt for 6 hours, then poured into 100 mL ether. The resulting precipitate is collected by suction filtration, rinsed with ether, and dried in vacuo to give compound 10 as a red powder.

The preceding examples can be repeated with similar success by substituting the specifically described carbocyanine compounds of the preceding examples with those generically and specifically described in the forgoing description. One skilled in the art can easily ascertain the essential characteristics of the present invention, and without departing from the spirit and scope thereof, can make various changes and modifications of the invention to adapt to various usages and conditions.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

1. A method for determining the presence or absence of an anionic protein and the presence or absence of a non-anionic protein in a sample, the method comprising:
a) contacting the sample with a dye to form a labeled sample, wherein the dye has the following formula: 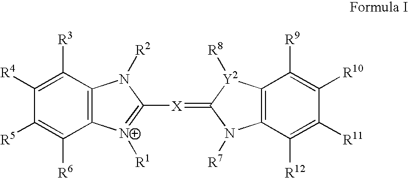
in which X is 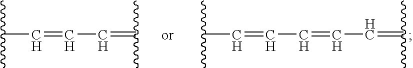
wherein at most one of the H is replaced with 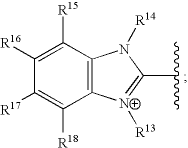
wherein
R¹ is hydrogen, alkyl or substituted alkyl;
R² is hydrogen, alkyl or substituted alkyl;
R⁷ is hydrogen, alkyl or substituted alkyl;
R⁸ is hydrogen, alkyl or substituted alkyl;
R¹³ is hydrogen, alkyl or substituted alkyl;
R¹⁴ is hydrogen, alkyl or substituted alkyl; and
R³ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁴ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁵ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁶ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁰ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹¹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹² is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁵ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁶ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁷ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁸ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl; or,
a member independently selected from
R³ in combination with R⁴;
R⁴ in combination with R⁵;
R⁵ in combination with R⁶;
R⁹ in combination with R¹⁰;
R¹⁰ in combination with R¹¹;
R¹¹ in combination with R¹²;
R¹⁵ in combination with R¹⁶;
R¹⁶ in combination with R¹⁷;
R¹⁷ in combination with R¹⁸
together with the atoms to which they are joined, form a ring which is a 5-, 6- or 7-membered cycloalkyl, a substituted 5-, 6- or 7-membered cycloalkyl, a 5-, 6- or 7-membered heterocycloalkyl, a substituted 5-, 6- or 7-membered heterocycloalkyl, a 5-, 6- or 7-membered aryl, a substituted 5-, 6- or 7-membered aryl, a 5-, 6- or 7-membered heteroaryl, or a substituted 5-, 6- or 7-membered heteroaryl;
b) incubating the labeled sample for sufficient time to allow the dye to associate with an anionic protein and non-anionic protein to form an incubated sample;
c) illuminating the incubated sample with a first appropriate wavelength for observing the anionic proteins to form a first illuminated sample;
d) illuminating the incubated sample with a second appropriate wavelength for observing non-anionic proteins to form a second illuminated sample;
e) observing the first and the second illuminated sample whereby the presence of anionic and non-anionic proteins are determined.

2. The method according to claim 1, wherein the dye has the formula: 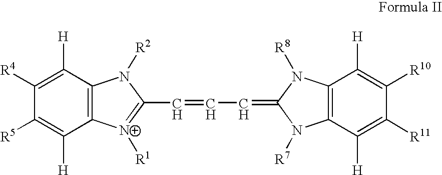
wherein R¹ is substituted alkyl or unsubstituted alkyl;
R² is substituted alkyl or unsubstituted alkyl;
R⁷ is substituted alkyl or unsubstituted alkyl;
R⁸ is substituted alkyl or unsubstituted alkyl; and
R⁴ is a halogen;
R⁵ is a halogen;
R¹⁰ is a halogen; and,
R¹¹ is a halogen.

3. The method according to claim 1, wherein the dye is:

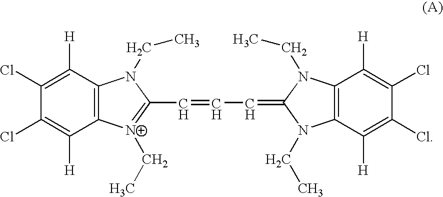

4. The method according to claim 1, wherein the dye further comprises a reactive group, carrier molecule or solid support.

5. The method according to claim 4, wherein the reactive group is an acrylamide, an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine, a hydrazide, an imido ester, an isocyanate, an isothiocyanate, a maleimide, a phosphoramidite, a reactive platinum complex, a sulfonyl halide, a thiol group, or a photoactivatable group.

6. The method according to claim 4, wherein the reactive group is carboxylic acid, succinimidyl ester of a carboxylic acid, hydrazide, amine or a maleimide.

7. The method according to claim 4, wherein the carrier molecule is an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus, or combinations thereof.

8. The method according to claim 4, wherein the carrier molecule is an antibody or fragment thereof, an avidin or streptavidin, a biotin, a blood component protein, a dextran, an enzyme, an enzyme inhibitor, a hormone, an IgG binding protein, a fluorescent protein, a growth factor, a lectin, a lipopolysaccharide, a microorganism, a metal binding protein, a metal chelating moiety, a non-biological microparticle, a peptide toxin, a phosphotidylserine-binding protein, a structural protein, a small-molecule drug, or a tyramide.

9. The method according to claim 4, wherein the solid support is a microfluidic chip, a silicon chip, a microscope slide, a microplate well, a silica gel, a polymeric membrane, a particle, a derivatized plastic film, a glass bead, cotton, a plastic bead, an alumina gel, a polysaccharide, polyvinylchloride, polypropylene, polyethylene, nylon, latex bead, magnetic bead, paramagnetic bead, or superparamagnetic bead.

10. The method according to claim 4, wherein the solid support is Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose or starch.

11. The method according to claim 1, wherein said sample is in a cuvette.

12. The method according to claim 1, wherein the sample is immobilized on a solid or semi solid support.

13. The method according to claim 12, wherein the solid or semi-solid support is a polymeric microparticle, polymeric membrane, polymeric gel or glass slide.

14. The method according to claim 1, wherein the anionic protein is phosphoproteins, calcium-binding proteins, sulfoproteins, or sialoglycoproteins.

15. A method for detecting the presence or absence of an anionic protein in a sample, the method comprising the steps of:
a) contacting the sample with a dye to form a labeled sample, wherein the dye is: 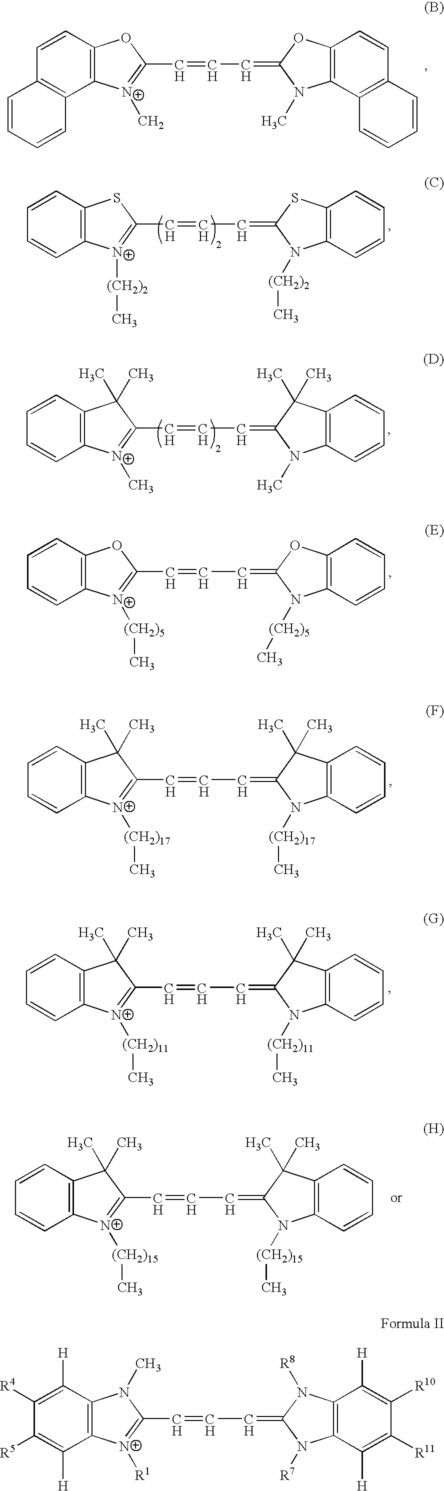
wherein R¹ is a substituted alkyl or unsubstituted alkyl;
R² is a substituted alkyl or unsubstituted alkyl;
R⁷ is a substituted alkyl or unsubstituted alkyl;
R⁸ is a substituted alkyl or unsubstituted alkyl; and,
R⁴ is a halogen;
R⁵ is a halogen;
R¹⁰ is a halogen; and
R¹¹ is a halogen;
b) incubating the labeled sample for a sufficient amount of time to allow the dye to associate with the anionic proteins to form an incubated sample;
c) illuminating the incubated sample with an appropriate wavelength to form an illuminated sample;
d) observing the illuminated sample whereby the presence or absence of the anionic proteins is determined.

16. The method according to claim 15, wherein the dye is:

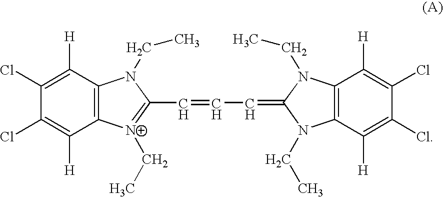

17. The method of claim 15, wherein said sample is in a cuvette.

18. The method according to claim 11, wherein the sample is immobilized on a solid or semi solid support.

19. The method according to claim 18, wherein the solid or semi-solid support is a polymeric microparticle, polymeric membrane, polymeric gel or glass slide.

20. The method according to claim 15, wherein the anionic protein is phosphoproteins, calcium-binding proteins, sulfoproteins, or sialoglycoproteins.

21. A kit which comprises:
a) a compound having the formula: 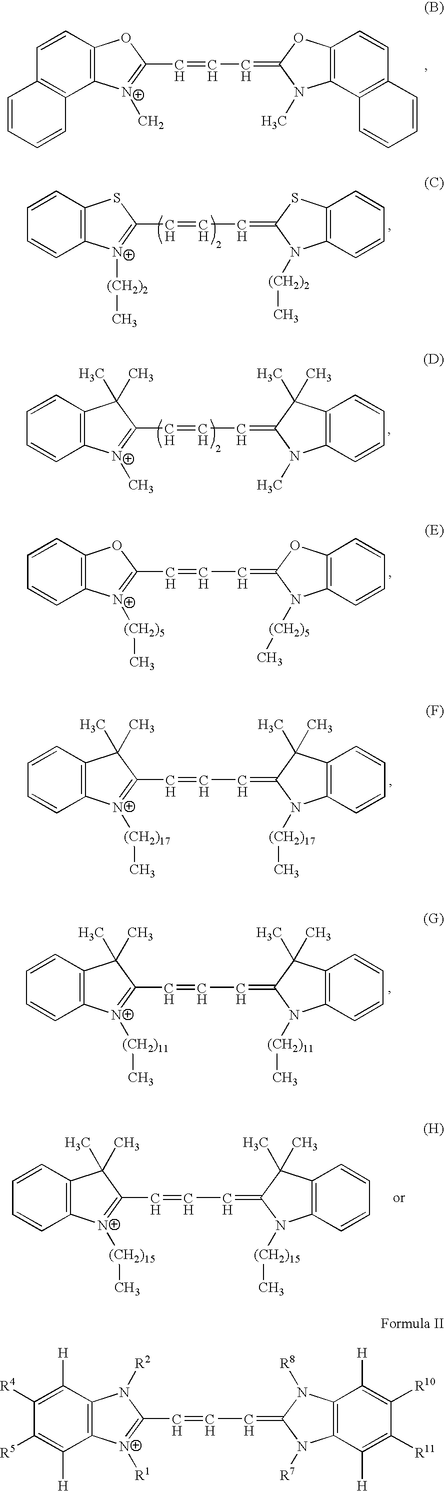
wherein R¹ is a substituted alkyl or unsubstituted alkyl;
R² is a substituted alkyl or unsubstituted alkyl;
R⁷ is a substituted alkyl or unsubstituted alkyl;
R⁸ is a substituted alkyl or unsubstituted alkyl; and,
R⁴ is a halogen;
R⁵ is a halogen;
R¹⁰ is a halogen; and
R¹¹ is a halogen; and,
b) instructions for detecting the presence or absence of anionic proteins in a sample.

22. A compound having the formula: 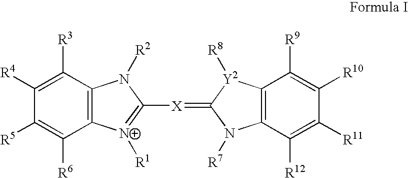
in which X is 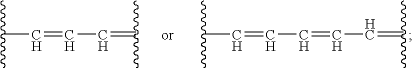
wherein at most one of the H is replaced with 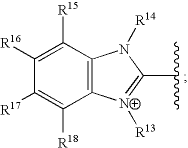
wherein
R¹ is hydrogen, alkyl or substituted alkyl;
R² is hydrogen, alkyl or substituted alkyl;
R⁷ is hydrogen, alkyl or substituted alkyl;
R⁸ is hydrogen, alkyl or substituted alkyl;
R¹³ is hydrogen, alkyl or substituted alkyl;
R¹⁴ is hydrogen, alkyl or substituted alkyl; and
R³ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁴ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁵ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁶ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R⁹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁰ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹¹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹² is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁵ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁶ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁷ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁸ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl;
R¹⁹ is hydrogen, OH, NH₂, NO₂, —SO₂NH₂, nitro, cyano, halogen, substituted alkyl, unsubstituted alkyl, substituted heteroalkyl, unsubstituted heteroalkyl, substituted 3- to 7-membered cycloalkyl, unsubstituted 3- to 7-membered cycloalkyl, substituted 5- to 7-membered heterocycloalkyl, unsubstituted 5- to 7-membered heterocycloalkyl, substituted aryl, unsubstituted aryl, substituted heteroaryl, or unsubstituted heteroaryl; or,
a member independently selected from
R³ in combination with R⁴;
R⁴ in combination with R⁵;
R⁵ in combination with R⁶;
R⁹ in combination with R¹⁰;
R¹⁰ in combination with R¹¹;
R¹¹ in combination with R¹²;
R¹⁵ in combination with R¹⁶;
R¹⁶ in combination with R¹⁷;
R¹⁷ in combination with R¹⁸
together with the atoms to which they are joined, form a ring which is a 5-, 6- or 7-membered cycloalkyl, a substituted 5-, 6- or 7-membered cycloalkyl, a 5-, 6- or 7-membered heterocycloalkyl, a substituted 5-, 6- or 7-membered heterocycloalkyl, a 5-, 6- or 7-membered aryl, a substituted 5-, 6- or 7-membered aryl, a 5-, 6- or 7-membered heteroaryl, or a substituted 5-, 6- or 7-membered heteroaryl;
with the proviso that at least one of R¹, R², R³, R⁴, R⁵, R⁶, R⁷, R⁸, R⁹, R¹⁰, R¹¹ or R¹² is a reactive group.
